(19) United States
(12) Patent Application Publication
(10) Pub. No.: US 2013/0243817 A1
(43) Pub. Date: Sep. 19, 2013

(54) VACCINE COMPOSITIONS

(71) Applicants: Ulf Schröder, Sundbyberg (SE); Hans Arwidsson, Strangnas (SE)

(72) Inventors: Ulf Schröder, Sundbyberg (SE); Hans Arwidsson, Strangnas (SE)

(73) Assignee: EUROCINE VACCINES AB, Stockholm (SE)

(21) Appl. No.: 13/821,929

(22) PCT Filed: Sep. 30, 2011
(86) PCT No.: PCT/EP11/67080

§ 371 Date: May 29, 2013

(30) Foreign Application Priority Data
Sep. 30, 2010 (DK) PA 2010 00887

(51) Int. Cl. A61K 39/39

(52) U.S. Cl. CPC A61K 39/39
USPC 424/209.1; 424/278.1; 424/238.1

(57) ABSTRACT

Present invention relates to vaccine formulations and adjuvants for use in e.g. compositions, thereby avoiding the phenomenon of Bell's palsy in a frequency above the natural occurance.

### FIELD OF THE INVENTION

The present invention relates to the use of adjuvants and vaccine compositions comprising adjuvants according to the invention, thereby avoiding the phenomenon of Bell's palsy in a frequency above the natural occurrence.

### BACKGROUND OF THE INVENTION

Bell's palsy is a dysfunction of cranial nerve VII (the facial nerve) that results in inability to control facial muscles on the affected side. Several conditions can cause a facial paralysis, e.g., brain tumor, stroke, and Lyme disease. However, if no specific cause can be identified, the condition is known as Bell's palsy. Named after Scottish anatomist Charles Bell, who first described it, Bell's palsy is the most common acute mononeuropathy (disease involving only one nerve) and is the most common cause of acute facial nerve paralysis.

Bell's palsy is defined as an idiopathic unilateral facial nerve paralysis, usually self-limiting and occurs in a frequency of about 30-35/100.000 annually. The typical characteristics are rapid onset of partial or complete palsy, usually in a single day. It can occur bilaterally resulting in total facial paralysis in around 1% of cases. In e.g. Europe the annual incidence of Bell palsy is approximately 32 cases per 100,000 persons. The right hand side is affected 63% of the time. Persons with diabetes have a 29% higher risk of being affected by Bell palsy than persons without diabetes. Thus, measuring blood glucose levels at the time of diagnosis of Bell palsy may detect undiagnosed diabetes. Internationally, the highest incidence was found in a study in Seckori, Japan, in 1986 and the lowest incidence was found in Sweden in 1971. Most population studies generally show an annual incidence of 30-35 cases per 100,000 population and year with an age-dependent incidence where older have a higher incidence (up to 60 cases per 100.000), and younger have a lower

It is thought that an inflammatory condition leads to swelling of the facial nerve. The nerve travels through the skull in a narrow bone canal beneath the ear. Nerve swelling and compression in the narrow bone canal are thought to lead to nerve inhibition, damage or death.

Bell's palsy is characterized by facial drooping on the affected half, due to malfunction of the facial nerve (VII cranial nerve), which controls the muscles of the face. Facial palsy is typified by inability to control movement in the facial muscles. The paralysis is of the infranuclear/lower motor neuron type.

The facial nerves control a number of functions, such as blinking and closing the eyes, smiling, frowning, lacrimation, and salivation. They also innervate the stapedial (stapes) muscles of the middle ear and carry taste sensations from the anterior two thirds of the tongue.

Due to an anatomical peculiarity, forehead muscles receive innervation from both sides of the brain. The forehead can therefore still be wrinkled by a patient whose facial palsy is caused by a problem in one of the hemispheres of the brain (central facial palsy). If the problem resides in the facial nerve itself (peripheral palsy) all nerve signals are lost on the ipsilateral (same side of the lesion) half side of the face, including to the forehead (contralateral forehead still wrinkles).

One disease that may be difficult to exclude in the differential diagnosis is involvement of the facial nerve in infections with the herpes zoster virus. The major differences in this condition are the presence of small blisters, or vesicles, on the external ear and hearing disturbances, but these findings may occasionally be lacking (zoster sine herpete).

Lyme disease may produce the typical palsy, and may be easily diagnosed by looking for Lyme-specific antibodies in the blood. In endemic areas Lyme disease may be the most common cause of facial palsy.

The degree of nerve damage can be assessed using the House-Brackmann score.

Although defined as a mononeuritis (involving only one nerve), patients diagnosed with Bell's palsy may have “myriad neurological symptoms” including “facial tingling, moderate or severe headache/neck pain, memory problems, balance problems, ipsilateral limb paresthesias, ipsilateral limb weakness, and a sense of clumsiness” that are “unexplained by facial nerve dysfunction”. This is yet an enigmatic facet of this condition.

### DETAILED DESCRIPTION OF THE INVENTION

During clinical trials of vaccine compositions there have been incidences of a heightened frequency of Bells palsy (Plos One (2009), vol 4 (9), e6999). Even though Bells palsy is usually a temporary condition that receives full restoration of nervous facial capacity, this is seen as a highly unacceptable side effect where, as a consequence thereof, the clinical trials are stopped and the vaccine never meets the market. It is contemplated that by using an adjuvant according to the invention, the unwanted side effect observed as Bell's palsy is avoided in the sense that the incidence or frequency of Bells palsy is the same as the natural incidence (15-30 cases per 100.000 individuals annually).

The adjuvant according to the invention may comprise

i) one or more carboxylic acids or one or more amines and optionally
ii) one or more monoglycerides.

An adjuvant according to the invention may also be an adjuvant comprising one amine or a mix of two amines.

An adjuvant according to the invention may also be an adjuvant comprising one amine or a mix of two amines and further comprising additional adjuvants such as e.g. squalene or soy bean oil. An adjuvant according to the invention may also be an adjuvant comprising

i) one or more carboxylic acids and optionally
ii) one or more mono-glycerides

An adjuvant according to the invention may also be an adjuvant comprising

i) one or more carboxylic acids and optionally
ii) one or more mono-glycerides and further comprising additional adjuvants such as e.g. squalene or soy oil.

An adjuvant according to the invention may also be an adjuvant comprising

i) one carboxylic acid or a mix of two carboxylic acids and optionally
ii) one or more mono-glycerides and further comprising additional adjuvants such as e.g. squalene or soy oil.

The invention also relates to the use of an adjuvant in a vaccine, the adjuvant comprising

i) one or more carboxylic acids and/or one or more amines and
b) optionally one or more mono-glycerides
and thereby reducing the risk of Bell's palsy in a subject to about the natural incidence.

Without being bound to any theory it is believed that due to the endogenous-like character of the, carboxylic acids (as well as their corresponding amines) and mono-glycerides according to the invention, a minimal systemic perturbation is accomplished which may in turn result in the low occurrence of observed cases of Bell's Palsy, i.e. an occurrence in about the same frequency of the naturally occurring cases of Bell's Palsy.

Moreover, it has also been speculated that one possible cause of Bell's Palsy is morphological changes of the olfactory nerve, which has been observed when i.a. cholera toxin has been used as an adjuvant in a vaccine.

Amines used in present invention comprise branched or unbranched, cyclic or acyclic, substituted or unsubstituted alkyl, alkenyl and alkynyl amines, optionally having multiple unsaturation (double or triple bond) which may further optionally be of different kind, of from 4 to 30 carbon atoms, such as, e.g., from 6 to 24 carbon atoms, from 8 to 20 carbon atoms or from 12 to 20 carbon atoms. It is also understood that the definition is intended to also cover different types of isomers such as e.g. diastereoisomerism (cis-trans isomers), which may be in any combination.

Carboxylic acids used in the present invention comprise long chain (C4-C30) alkyl, alkenyl or alkynyl carboxylic acids which may optionally be branched or unbranched, cyclic or acyclic, optionally having multiple unsaturation (double or triple bond) which may further optionally be of different kind.

Monoglycerides used in the present invention may be carboxylic acid esters of glycerin, wherein the carboxylic acids may be long chain (C4-C30) alkyl, alkenyl or alkynyl carboxylic acids which may optionally be branched or unbranched, optionally having multiple unsaturation (double or triple bond) which may further optionally be of different kind.

### DEFINITIONS

Throughout the text including the claims, the following terms shall be defined as indicated below.

The term “amine” encompasses branched or unbranched, cyclic or acyclic, substituted or unsubstituted alkyl, alkenyl and alkynyl amines, optionally having multiple unsaturation (double or triple bond) which may further optionally be of different kind, of from 4 to 30 carbon atoms, such as, e.g., from 6 to 24 carbon atoms, from 8 to 20 carbon atoms or from 12 to 20 carbon atoms. It is also understood that the definition is intended to also cover different types of diastereoisomerism (cis-trans isomers), which may be in any combination.

Examples are, but not limited to; lauryl amine (C12), myristyl amine (C14), cetyl amine (C16), palmitoleyl amine (C16:1), oleyl amine (C18:1), linoleyl amine (C18:2) and sterayl amine (C18). Other examples are hexyl amine, octyl amine, decyl amine, undecyl amine, dodecyl amine

The term “carboxylic acid” encompasses branched or unbranched, cyclic or acyclic, substituted or unsubstituted alkyl, alkenyl and alkynyl carboxylic acids, optionally having multiple unsaturation (double or triple bond) which may further optionally be of different kind (double and triple bonds in any mix or combination), of from 4 to 30 carbon atoms, such as, e.g., from 6 to 24 carbon atoms, from 8 to 20 carbon atoms or from 12 to 20 carbon atoms. It is also understood that the definition is intended to also cover different types of diastereoisomerism (cis-trans isomers), which may be in any combination.

Examples are, but not limited to; lauric acid (C12), myristic acid (C14), palmitic acid (C16), palmitoleic acid (C16:1), oleic acid (C18:1), linoleic acid (C18:2) and stearic acid. Other examples are hexanoic acid, caprylic acid, decanoic acid (capric acid), arachidic acid, behenic acid, lignoceric acid, alpha-linolenic acid, stearidonic acid, eicosapentaenoic acid, docosahexaenoic acid, gamma-linolenic acid, dihomo-gamma-linolenic acid, arachidonic acid, erucic acid and nervonic acid.

The term “monoglyceride” encompasses carboxylic acid mono-esters of glycerine (propane-1,2,3-triol) wherein the carboxylic acid may be branched or unbranched, cyclic or acyclic, substituted or unsubstituted alkyl, alkenyl and alkynyl carboxylic acids, optionally having multiple unsaturation (double or triple bond) which may further optionally be of different kind, of from 4 to 30 carbon atoms, such as, e.g., from 6 to 24 carbon atoms, from 8 to 20 carbon atoms or from 12 to 20 carbon atoms. It is also understood that the definition is intended to also cover different types of diastereoisomerism (cis-trans isomers), which may in any combination.

Examples of acids used in the esterification of glycerol are, but not limited to, lauric acid (C12), myristic acid (C14), palmitic acid (C16), palmitoleic acid (C16:1), oleic acid (C18:1), linoleic acid (C18:2) and stearic acid. Other examples are hexanoic acid, caprylic acid, decanoic acid (capric acid), arachidic acid, behenic acid, lignoceric acid, alpha-linolenic acid, stearidonic acid, eicosapentaenoic acid, docosahexaenoic acid, gamma-linolenic acid, dihomo-gamma-linolenic acid, arachidonic acid, erucic acid and nervonic acid.

The term “antigen” is defined as anything that can serve as a target for an immune response. The immune response can be either cellular or humoral and be detected in systemic and/or mucosal compartments.

The term “vaccine” is defined herein as a suspension or solution of antigenic moieties, usually consisting of infectious agents, or some part of the infectious agents, that is introduced to an animal or human body to produce active immunity.

The term “adjuvant” or “adjuvant mix” herewith used interchangeably and as used herein is any substance whose admixture with an administered immunogen increases or otherwise modifies the immune response upon introduction into the human or animal body.

The term “medium” as used herein is intended to mean physiologically acceptable medium, such as e.g an aqueous medium which may contain buffers, salts, pH-regulators, preservatives etc.

The term “reducing” as used in reducing the incidence of a condition such as e.g. reducing the incidence of Bell's palsy is intended to mean a reduction of the incidence to a level about equal to the natural incidence of the condition. In case of Bell's palsy the natural incidence is 30-35 persons afflicted with Bell's palsy per 100.000 persons annually.

The term “avoiding” an incidence of a condition is intended to mean that a subject will have a risk of attracting a condition, such as e.g. Bell's palsy, about equal to the naturally occurring frequency of the condition. With regards to Bell's palsy, a subject will have a risk of about 30-35 cases per 100.000 individuals annually of acquiring Bell's palsy and consequently with the respect to this invention a subject avoiding the condition of Bell's palsy is means that the subject will have about the same risk of acquiring Bell's palsy as the natural incidence of 30-35 cases per 100.000 individuals annually.

The amine or amines in the adjuvant mix used according to the invention may be branched or unbranched, cyclic or acyclic, substituted or unsubstituted alkyl, alkenyl and alkynyl amines, optionally having multiple unsaturation (double or triple bond) which may further optionally be of different kind, of from 4 to 30 carbon atoms, such as, e.g., from 6 to 24 carbon atoms, from 8 to 20 carbon atoms or from 12 to 20 carbon atoms. The amines may also be different diastereoisomers, cis or trans, which may further be (in the case of multiple unsaturation) of different kind in the same molecule. The diastereomers may also be present in any mix, such as e.g. 50% cis and 50% trans, or 40% cis and 60% trans, or 30% cis and 70% trans, or 20% cis and 80% trans, or 10% cis and 90% trans, or 5% cis and 95% trans or 1% cis and 99% trans, or 40% trans and 60% cis, or 30% trans and 70% cis, or 20% trans and 80% cis, or 10% trans and 90% cis, or 5% trans and 95% cis or 1% trans and 99% cis, or 100% cis and 0% trans, or 0% cis and 100% trans.

Examples are, but not limited to, lauryl amine (C12), myristyl amine (C14), cetyl amine (C16), palmitoleyl amine (C16:1), oleyl amine (C18:1), linoleyl amine (C18:2) and sterayl amine (C18). Other examples are hexyl amine, octyl amine, decyl amine, undecyl amine, dodecyl amine

An adjuvant according to the invention comprising two amines may be such that the w/w ratio between the two amines is from about 0.1 to about 10, such as, e.g., from about 0.25 to about 9, from about 0.5 to about 8, from about 0.75 to about 7, from about 1 to about 6, from about 1 to about 5, from about 1 to about 4, from about 1 to about 3, from about 1 to about 2 or from about 1 to about 1. Alternatively, the total amount of one or more amines in the adjuvant may range e.g. from about 100% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 9% or less, about 8% or less, about 7% or less, about 6% or less, about 5% or less, about 4% or less, about 3% or less, about 2% or less, about 1% or less, about 0.5% or less, about 0.25% or less, about 0.1% or less, about 0.05% or less of the volume of the adjuvant

An adjuvant according to the invention comprising two amines may be a mixture of oeyl amine and lauryl amine. The w/w ratio of oeyl amine to lauryl amine may be from about 0.1 to about 10, such as, e.g., from about 0.25 to about 9, from about 0.5 to about 8, from about 0.75 to about 7, from about 1 to about 6, from about 1 to about 5, from about 1 to about 4, from about 1 to about 3, from about 1 to about 2 and from about 1 to about 1.

The carboxylic acids used in the adjuvant mix may be branched or unbranched, cyclic or acyclic, substituted or unsubstituted alkyl, alkenyl and alkynyl carboxylic acids, optionally having multiple unsaturation (double or triple bond) which may further optionally be of different kind (double and triple bonds in any mix or combination), of from 4 to 30 carbon atoms, such as, e.g., from 6 to 24 carbon atoms, from 8 to 20 carbon atoms or from 12 to 20 carbon atoms. The carboxylic acids may also be different diastereoisomers, cis or trans, which may further be (in the case of multiple unsaturation) of different kind in the same molecule. The diastereomers may also be present in any mix, such as e.g. 50% cis and 50% trans, or 40% cis and 60% trans, or 30% cis and 70% trans, or 20% cis and 80% trans, or 10% cis and 90% trans, or 5% cis and 95% trans or 1% cis and 99% trans, or 40% trans and 60% cis, or 30% trans and 70% cis, or 20% trans and 80% cis, or 10% trans and 90% cis, or 5% trans and 95% cis or 1% trans and 99% cis, or 100% cis and 0% trans, or 0% cis and 100% trans.

Examples are, but not limited to, lauric acid (C12), myristic acid (C14), palmitic acid (C16), palmitoleic acid (C16:1), oleic acid (C18:1), linoleic acid (C18:2) and stearic acid. Other examples are hexanoic acid, caprylic acid, decanoic acid (capric acid), arachidic acid, behenic acid, lignoceric acid, alpha-linolenic acid, stearidonic acid, eicosapentaenoic acid, docosahexaenoic acid, gamma-linolenic acid, dihomo-gamma-linolenic acid, arachidonic acid, erucic acid and nervonic acid.

The monoglycerides according to the invention have the formula

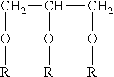

wherein R is selected from H and an acyl radical containing from 4 to 30 carbon atoms with the proviso that two of the R groups are H. In a mono-glyceride the acyl chains are normally placed on carbon atom 1 or 3 of the glycerol backbone, but there will often be a acyl migration between the carbon atoms 1 and 3, and the center carbon atom 2, resulting in that approximately 90% of the acyl chains will be positioned on the carbon atom 1 or 3, and about 10% will be positioned on the center carbon atom. The acyl radical may be, but is not limited to, a selection from the corresponding carboxylic acids used in the esterification of glycerol and may be branched or unbranched, cyclic or acyclic, substituted or unsubstituted alkyl, alkenyl and alkynyl carboxylic acids, optionally having multiple unsaturation (double or triple bond) which may further optionally be of different kind, of from 4 to 30 carbon atoms, such as, e.g., from 6 to 24 carbon atoms, from 8 to 20 carbon atoms or from 12 to 20 carbon atoms. The carboxylic acids used in the esterification of glycerol may also be different diasetreoisomers, cis or trans, which may further be (in the case of multiple unsaturation) of different kind in the same molecule. The diastereomers may also be present in any mix, such as e.g. 50% cis and 50% trans, or 40% cis and 60% trans, or 30% cis and 70% trans, or 20% cis and 80% trans, or 10% cis and 90% trans, or 5% cis and 95% trans or 1% cis and 99% trans, or 40% trans and 60% cis, or 30% trans and 70% cis, or 20% trans and 80% cis, or 10% trans and 90% cis, or 5% trans and 95% cis or 1% trans and 99% cis, or 100% cis and 0% trans, or 0% cis and 100% trans.

Examples of carboxylic acids used in the mono-esterification of glycerol are, but not limited to, lauric acid (C12), myristic acid (C14), palmitic acid (C16), palmitoleic acid (C16:1), oleic acid (C18:1), linoleic acid (C18:2) and stearic acid. Other examples are hexanoic acid, caprylic acid, decanoic acid (capric acid), arachidic acid, behenic acid, lignoceric acid, alpha-linolenic acid, stearidonic acid, eicosapentaenoic acid, docosahexaenoic acid, gamma-linolenic acid, dihomo-gamma-linolenic acid, arachidonic acid, erucic acid and nervonic acid.

In the present invention is used distilled 1-monoglyceride with a purity of at least 80% w/w, such as, e.g., at least 90% w/w or at least 95% w/w.

The concentration of monoglyceride may be in the range of e.g. about 0.1 g to about 50 g per 100 ml of adjuvant mix, preferably in the range of e.g. about 1 g about 20 g per 100 ml, or about 0.5 g to about 40 g, such as e.g. 0.5 g to about 30 g, such as about e.g. 0.5 g to about 25 g, such as e.g. 1 g to about 20 g, such as about 2 g to about 15 g, such as e.g. 5 g to about 10 g per 100 ml of adjuvant mix.

The carboxylic acid concentration may be in the range of e.g. about 0.1-50 g per 100 ml of adjuvant mix, preferably in the range of 1-20 g per 100 ml of adjuvant mix or about 0.5 g to about 40 g, such as e.g. 0.5 g to about 30 g, such as about e.g. 0.5 g to about 25 g, such as e.g. 1 g to about 20 g, such as about 2 g to about 15 g, such as e.g. 5 g to about 10 g per 100 ml of adjuvant mix.

In an adjuvant mix between one or more monoglycerides and one or more carboxylic acids the percentage of monoglyceride in carboxylic acid may be varied between 1 to 99%, preferably between 10-90%, or such as e.g. the total amount of the one or more mono-glycerides in the adjuvant may range e.g. from about 100% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 9% or less, about 8% or less, about 7% or less, about 6% or less, about 5% or less, about 4% or less, about 3% or less, about 2% or less, about 1% or less, about 0.5% or less, about 0.25% or less, about 0.1% or less, about 0.05% or less.

Accordingly the total amount of one or more carboxylic acids in the adjuvant may range e.g. from about 100% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 9% or less, about 8% or less, about 7% or less, about 6% or less, about 5% or less, about 4% or less, about 3% or less, about 2% or less, about 1% or less, about 0.5% or less, about 0.25% or less, about 0.1% or less, about 0.05% or less of the volume of the adjuvant.

The adjuvant mix according to the invention comprises the adjuvant components, i.e. one or more carboxylic acids either alone or mixed with one or more monoglycerides in a concentration that elicits an immune response in a human or animal to an antigen administered to the human or animal when an antigen is added. The inventors of present invention have found that adjuvants according to present invention are particularly useful when vaccination is performed via the nasal route, e.g. administration to the mucosa of the nasal cavity. The inventors have surprisingly found that use of adjuvants according to present invention in vaccination via the nasal route improves the immune response upon vaccination.

As mentioned above, the adjuvant mix may comprise one or more carboxylic acid and one or mono-glycerides. The total concentration of carboxylic acid together with one or more monoglycerides in an adjuvant mix is at the most 75% w/v, or at the most 50% w/v, or at the most 25% w/v, or at the most 20% w/v, or at the most 15% w/v, or at the most 10% w/v, or at the most 5% w/v, or at the most 4% w/v, or at the most 3% w/v, or at the most 2% w/v or at the most 1% w/v or at the most 0.5% w/v or at most 0.1% w/v of the total volume of the adjuvant.

Furthermore, the adjuvant mix comprising one or more monoglycerides together with one or more carboxylic acids, may have the total amount of the one or more monoglycerides together with the one or more carboxylic acids in an adjuvant mix is in range from about 0.1% w/v to about 10% w/v, such as, e.g., from about from about 0.25% w/v to about 9% w/v, from about 0.5% w/v to about 8% w/v, from about 1% w/v to about 7% w/v, from about 1% w/v to about 6% w/v, from about 1% w/v to about 5% w/v, from about 1% w/v to about 4% w/v, from about 1% w/v to about 3% w/v, from about 1% w/v to about 2% w/v or from about 0.5% w/v to about 4% w/v of the total volume of the adjuvant mix

The adjuvant according to the invention may further comprise a medium such as e.g. surface-active agents, which may be hydrophilic and inert and biocompatible, such as, e.g., poloxamers such as e.g. Pluronic F68 or Pluronic-127.

The medium may further comprise one or more physiologically acceptable additives or pharmaceutical excipients, such as, e.g., buffering agents, such as, e.g. Tris, stabilizing agents, osmotically active agents, preservatives and pH adjusting agents.

The pH of the medium should be within the physiologically acceptable range, such as e.g. pH 4 to about pH 9, such as from e.g. about pH 5 to about pH 7, such as e.g. about pH 7 to about pH 9, such as e.g. about 7.5 to about pH 8.5 or such as from e.g. about pH 5.5 to about pH 6.5, or such as e.g. about pH 6 or about pH 5 or about pH 8.

The adjuvant according to the invention may also further comprise additional adjuvants. Additional adjuvants may be e.g., squalene, an aluminum salt such as e.g. aluminum hydroxide, aluminum phosphate, aluminum hydroxyphosphate sulfate, aluminum potassium sulfate, soy bean oil or any combination thereof. The amount of the additional adjuvant may be in the range of e.g. about 0.1-50 g per 100 ml of adjuvant mix, preferably in the range of 1-20 g per 100 ml of adjuvant mix or about 0.5 g to about 40 g, such as e.g. 0.5 g to about 30 g, such as about e.g. 0.5 g to about 25 g, such as e.g. 1 g to about 20 g, such as about 2 g to about 15 g, such as e.g. 5 g to about 10 g per 100 ml of adjuvant mix.

The final concentration of the one or more carboxylic acids optionally together with one or more mono-glycerides or may be up to about 10% of the final adjuvant or vaccine composition, such as e.g. up to about 8%, such as e.g. up to about 7%, such as e.g. up to about 5%, such as e.g. up to about 3%, such as e.g. up to about 1%, such as e.g. up to about 0.1% of the final adjuvant or vaccine composition.

The final concentration of the one or more amines may be up to about 10% of the final adjuvant or vaccine composition, such as e.g. up to about 8%, such as e.g. up to about 7%, such as e.g. up to about 5%, such as e.g. up to about 3%, such as e.g. up to about 1%, such as e.g. up to about 0.1% of the final adjuvant or vaccine composition.

### Vaccines

An adjuvant mix according to the present invention is intended to be used for the preparation of a vaccine. Such a vaccine comprises the adjuvant together with an immunogenic quantity of an antigen component and, optionally dispersed in a medium such as an aqueous medium.

Consequently a vaccine composition according to the invention may comprise

i) one or more carboxylic acids or one or more amines and optionally
ii) one or more mono-glycerides
ii) one or more antigens

For example, the vaccine composition of the invention may comprise

i) from about 0.1 g to about 90 g or one or two carboxylic acids
ii) from about 0.01 g to about 90 g of antigen.
per 100 g of final vaccine composition.

More specifically so, the vaccine composition of the invention may comprise

i) from about 0.1 g to about 90 g carboxylic acid either as a single carboxylic acid or a mix of two acids
ii) from about 0.1 g to about 90 g monoglyceride
iii) from about 0.001 g to about 90 g of antigen.
per 100 g of final vaccine composition.

It is to be clearly understood that the above vaccine compositions may further comprise one or more additional adjuvants such as e.g. squalene or and oil such as e.g. soy bean oil.

Thus, the vaccine composition of the invention may comprise

i) from about 0.1 g to about 90 g carboxylic acid either as a single carboxylic acid or a mix of two acids
ii) from about 0.1 g to about 90 g monoglyceride
iii) from about 0.1 g to about 90 g of additional adjuvant such as e.g. squalene or soybean oil.
iv) from about 0.001 g to about 90 g of antigen.
per 100 g of final vaccine composition.

More specifically so, the vaccine composition of the invention may comprise

i) from about 0.1 g to about 90 g carboxylic acid
ii) from about 0.1 g to about 90 g monoglyceride
iii) from about 0.01 g to about 90 g of antigen.
per 100 g of final vaccine composition.

Moreover, the vaccine composition may comprise e.g.

i) from about 0.1 g to about 90 g of one or two or more amines
ii) from about 0.01 g to about 90 g of antigen.
per 100 g of final vaccine composition.

The vaccine composition may further comprise additional adjuvants such as squalene or an oil such as e.g. soy bean oil.

The antigens may be e.g. whole inactivated antigens such as e.g. whole inactivated viruses. The antigen may also be part of a pathogen such as e.g. part of an inactivated virus. The antigen components that may be used are, but not limited to, for example, viral, bacterial, mycobaterial or parasitic antigens. Viral pathogens are e.g. hepatitis viruses A, B, C, D & E3, HIV, herpes viruses 1, 2, 6 & 7, cytomegalovirus, varicella zoster, papilloma virus, Epstein Barr virus, influenza viruses, para-influenza viruses, adenoviruses, bunya viruses (e.g. hanta virus), coxsakie viruses, picorna viruses, rotaviruses, respiratory syncytial viruses, pox viruses, rhinoviruses, rubella virus, papovavirus, mumps virus and measles virus.

Bacterial pathogens may be e.g. Mycobacteria causing tuberculosis and leprosy, pneumocci, aerobic gram negative or gram-positive bacilli, mycoplasma, staphyloccocal infections, streptococcal infections, Helicobacter pylori, salmonellae and chlamydiae.

The amount of the one or more antigens may be in the range of e.g. from about 300 μg or less, about 200 μg or less, about 100 μg or less, about 95 μg or less, about 90 μg or less, about 85 μg or less, about 80 μg or less, about 75 μg or less, about 70 μg or less, about 65 μg or less, about 60 μg or less, about 55 μg or less, about 50 μg or less, about 45 μg or less, about 40 μg or less, about 35 μg or less, about 30 μg or less, about 25 μg or less, about 20 μg or less, about 15 μg or less, about 10 μg or less, about 9 μg or less, about 8 μg or less, about 7 μg or less, about 6 μg or less, about 5 μg or less, about 4 μg or less about 3 μg or less, about 2 μg or less, about 1 μg or less, about 0.5 μg or less, about 0.25 μg or less, about 0.1 μg or less, about or about 0.05 μg or less. The antigen may also be in any amount sufficient to elicit an immune response in the subject.

The vaccine formulation according to present invention may be used for protection or treatment of animals or humans against a variety of disease states such as, for example, viral, bacterial or parasitic infections, cancer, allergies and autoimmune disorders. Some specific examples of disorders or disease states, which can be protected against or treated by using the methods or compositions according to the present invention, are viral infections caused by hepatitis viruses A, B, C, D & E3, HIV, herpes viruses 1, 2, 6 & 7, cytomegalovirus, varicella zoster, papilloma virus, Epstein Barr virus, influenza viruses, para-influenza viruses, adenoviruses, bunya viruses (e.g. hanta virus), coxsakie viruses, picorna viruses, rotaviruses, respiratory syncytial viruses, pox viruses, rhinoviruses, rubella virus, papovavirus, mumps virus and measles virus.

The diseases may also be bacterial infections such as infections caused by Mycobacteria causing tuberculosis and leprosy, pneumocci, aerobic gram negative bacilli, mycoplasma, staphyloccocal infections, streptococcal infections, Helicobacter pylori, salmonellae, diphtheria and chlamydiae.

The diseases may also be parasitic malaria, leishmaniasis, trypanosomiasis, toxoplasmosis, schistosomiasis, filariasis or various types of cancer such as, e.g. breast cancer, stomach cancer, colon cancer, rectal cancer, cancer of the head and neck, renal cancer, malignant melanoma, laryngeal cancer, ovarian cancer, cervical cancer, prostate cancer.

The diseases may also be allergies due to house dust mite, pollen and other environmental allergens and autoimmune diseases such as, e.g. systemic lupus erythematosis.

The antigen in the vaccine composition may be whole inactivated antigens such as e.g. whole inactivated viruses. Inactivation processes are well known in the art such as heat inactivation, irradiation inactivation by UV-light or in activation by formalin inactivation or treatment with beta-propiolactone.

The vaccine composition according to the invention may further comprise pharmaceutically acceptable excipients such as e.g. a medium which may be an aqueous medium further comprising a surface-active agent, which may be hydrophilic and inert and biocompatible, such as, e.g., poloxamers such as e.g. Pluronic F68 or Pluronic 127.

A vaccine according to present invention may thus further comprise further adjuvants, antibacterial agents, antioxidants, viral inactivators, preservatives, dyes, stabilizers, anti-foaming agents, surfactants (non-ionic, anionic or cationic) or any combination thereof.

The pH of the vaccine mix should be within the physiologically acceptable range, such as e.g. from about pH 4 to about pH 9, such as from e.g. about pH 5 to about pH 7, such as e.g. about pH 7 to about pH 9, such as e.g. about 7.5 to about pH 8.5 or such as from e.g. about pH 5.5 to about pH 6.5, or such as e.g. about pH 6 or about pH 5 or about pH 8. It is envisaged that the pH of the adjuvant or vaccine composition in the case where amines are used will be in e.g. range of about pH 5 to about pH 7, such as about pH 5.5. In the case where adjuvants or vaccine compositions are based on carboxylic acids the pH range may be e.g. about pH 7.5 to about pH 8.5.

The additional adjuvant may be such as e.g., oils such as squalene or soybean oil or an aluminum salt such as e.g. aluminum hydroxide, aluminum phosphate, aluminum hydroxyphosphate sulfate, aluminum potassium sulfate or any combination thereof.

The antibacterial agents may be e.g. amphotericin or any derivative thereof, chlorotetracyclin, formaldehyde or formalin, gentamicin, neomycin, polymyxin B or any derivative thereof, streptomycin or any combination thereof.

The antioxidants may be e.g. ascorbic acid or tocopherol or any combination thereof.

The viral inactivators may be e.g. formalin, beta-propiolactone, UV-radiation, heating or any combination thereof.

The preservatives may be e.g. benzethonium chloride, EDTA, phenol, 2-phenoxyethanol or thimerosal or any combination thereof.

The dyes may be e.g. any indicators (such as e.g. phenol red) or brilliant green or any combination thereof.

The anti-foaming agents may be e.g. polydimethylsilozone.

The surfactants may be e.g. anionic, cationic or non-ionic or zwitterionic, such as e.g. polyoxyethylene and derivatives thereof, polysorbates (such as e.g. polysorbate 20 or polysorbate 80), Tween 80, poloxamers (such as e.g Pluronic F68) or any combination thereof.

The invention also relates to or enables prophylaxis and/or treatment of any infectious diseases as disclosed herein.

Normally, the vaccines may be administered in any convenient manner such as by parenteral or mucosal administration, such as, e.g. nasal, oral, rectal, vaginal, lung, aural, or topical administration, or by intravenous, intramuscular, subcutaneous, intradermal administration or topical routes by transdermal application by creams, ointments or trandermal pathches, and any combinations thereof.

The nose is a very attractive route for immunization via mucosal administration due to the fact that it is easily accessible, highly vascularized and contains a large absorptions surface. Both mucosal, systemic and cellular immune responses can be induced and immune response can be induced at distant mucosal sites, such as the vagina and rectum. Furthermore large populations can easily be immunized, with less risk of infection.

In case administration to the nose is targeted, the mode of administration can be e.g. by spraying the vaccine into the nasal cavity or by administering the vaccine via pipette by dripping the vaccine into the nasal cavity or onto the nasal mucosal wall.

Parenteral administration is envisaged to be intravenous, intraarterial, intramuscular, intracerebral, intracerebroventricular, intracardiac, subcutaneous, intraosseous, intradermal, intrathecal, intraperitoneal, intravesical or intracavernosal injection.

The invention thus relates to a method of enhancing an immune response in a human or animal to an antigen administered to the human or animal, the method comprising administering an immune response enhancing effective amount of a vaccine comprising the adjuvant according to the present invention to the human or animal. The method may further comprise administering to a subject a vaccine composition according to the invention via nasal, intravenous, subcutaneous or intramuscular administration.

### FIGURE LEGENDS

FIG. 1 Illustrates the number of study groups that reach individual EMA [European Medical Agency) HAI (hemagglutination-inhibition) criteria after each dose.

FIG. 2 Illustrates the combination dose-response score of negatively charged adjuvant formulation (HAI, N-IgA H1/Bri/Cal, H3/Bris/Cal, S-IgG, S-IgA and INF-γ).

The following examples are intended to illustrate the invention without limiting it in any way.

### EXAMPLES

### Example 1

### Adjuvant Preparation

A positively charged adjuvant preparation was obtained by mixing oeyl amine and lauryl amine in a ratio of 1:1 (w/w). The resulting mix was thereafter emulsified in 50 mM acetate buffer at pH 6.5 such that the final concentration of oeyl amine/lauryl amine was 2% w/v.

Alternatively, a positively charged adjuvant preparation was obtained by mixing oeyl amine, lauryl amine and squalene in a ratio of 1:1:1 (w/w). The resulting mix was thereafter emulsified in 50 mM acetate buffer at pH 6.5 such that the final concentration of oeyl amine/lauryl amine/squalene was 2% w/v.

Alternatively oeyl amine was emulsified in 50 mM acetate buffer at pH 6.5 such that the final concentration of oeyl amine was 2% w/v.

Alternatively, an oil, such as squalene could be added to the oeyl amine in a 1:1 ratio (w/w), thus providing a final concentration of oleylamine and squalene of 2% in the emulsion.

Given the use of acetate as the buffer, the pH of the final formulation for the positively charged formulations can be in the interval of pH 5-7.5.

The above positively charged emulsions were produced either by high pressure homogenization or ultrasound probe sonication. The resulting stock-solutions were optionally diluted in dilutions of 2, 4, 8, 16, and 32 with water or acetate buffer solution.

A negatively charged emulsion was produced by mixing oleic acid and mono-olein in a 1:1 ratio, wherafter the mixture was emulsified in a 0.1M Tris-buffer having a pH of 8.0.

Alternatively, a negatively charged emulsion was produced by mixing oleic acid and lauric acid in a 1:1 ratio, wherafter the mixture was emulsified in a 0.1M Tris-buffer having a pH of 8.0.

The final concentration of carboxylic acids optionally together with mono-glycerides may be up to 10% of the final adjuvant or vaccine composition.

The above negatively charged emulsions were produced either by high pressure homogenization or ultrasound probe sonication. The resulting stock-solutions were optionally diluted in dilutions of 2, 4, 8, 16, and 32 with water or Tris buffer solution.

A negatively charged adjuvant is prepared by mixing oleic acid (0.46 g) and lauric acid (0.34 g) which is subsequently sonicated with 9.2 ml of 0.1M Tris-buffer (pH 8.0). The pH of the final solution is adjusted to pH 8.0 with 5 M NaOH. The final concentration is 8% of the lipid formulation. The composition is henceforth abbreviated as Adjuvant A.

Furthermore, a further negatively charged adjuvant is prepared by mixing mono-olein (0.45 g) and oleic acid (0.35 g) and subsequently sonicating with 9.2 ml of 0.1M Tris buffer (pH 8.0). The final formulation is adjusted to pH 8 with 5M NaOH. The final concentration is 8% of the lipid formulation. The composition is henceforth abbreviated as Adjuvant B.

Yet a further adjuvant is prepared by mixing mono-olein (0.15 g), oleic acid (0.12 g) and soybean oil (0.53 g). The mixture is subsequently sonicated with 0.2 ml of 0.1M Tris buffer (pH 8.0). The final formulation is adjusted to pH 8 with 5M NaOH. The final concentration is 8% of the lipid formulation. The composition is henceforth abbreviated as Adjuvant C.

Vaccine formulations of the adjuvants are prepared by mixing the suitable antigen and the 8% lipid adjuvant formulation in a 1:1 ration, in order to have a final lipid concentration of 4% in each formulation.

The 8% lipid adjuvant formulations can of course be dilued into 1%, 2%, 4% or 6% solutions as desired by addition of further buffer solution.

### Example 1.1

A positively charged formulation with oeyl amine having a concentration of 2% was mixed at a 1+1 ratio (v/v) with a suspension of inactivated influenza virus particles (strain H1N1/California). The mixture was subsequently administered intranasally to mice in a 5 μl volume into each nostril. The dose of the influenza virus particles was the equivalent of 1.5 μg of hemeagglutinin (HA). The mice were immunized on three occasion, separated with three weeks. Three weeks after the last immunization the mice were killed and blood samples were analyzed for immunological response. The results showed that the HAI titer from the mice receiving the non-adjuvanted antigen gave rise to a HAI titer of 47 (geometrical mean, N=8), whereas the positively charged formulation gave rise to a HAI titer of 1140 (geometrical mean, N=8). Thus 24-fold increase of the HAI titer was obtained.

Performing assay of the T-cell response as pg/ml of I NF-γ after NP stimulation, revealed that the positively charged adjuvant enhanced the response 28-fold over the non-adjuvanted formulation.

The final concentration of amines may be up to 10% of the final adjuvant or vaccine composition

### Example 1.2

A positively charged formulation with oleylamine and squalene having a concentration of 2%, was mixed at a 1+1 ratio (v/v) with a suspension of inactivated influenza virus particles (strain H1N1/California). The mixture was subsequently administered intranasally to mice in a 5 μl volume into each nostril. The dose of the inactivated influenza virus particles was the equivalent of 1.5 μg of hemeagglutinin (HA). The mice were immunized on three occasions, separated with three weeks. Three weeks after the last immunization the mice killed and blood samples were analyzed for immunological T-cell response.

Performing the assay of the T-cell response as pg/ml of INF-γ after NP stimulation, revealed that the positively charged adjuvant enhanced the response 60-fold over the non-adjuvanted formulation.

### Example 2

A negatively charged formulation with oleic acid and mono-olein having a concentration of 4%, was mixed at a 1+1 ratio (v/v) with a suspension of inactivated influenza virus particles (strain H1N1/California). The mixture was subsequently administered intranasally to mice in a 5 μl volume into each nostril. The dose of the influenza virus particles was the equivalent of 1.5 μg of hemagglutinin (HA). The mice were immunized on three occasion, separated with three weeks. Three weeks after the last immunization the mice killed and blood samples were analyzed for immunological response.

The results showed that the HAI titer from the mice receiving the non-adjuvanted antigen gave rise to a HAI titer of 47 (geometrical mean, N=8), whereas the negatively charged formulation gave rise to a HAI titer of 147 (geometrical mean, N=8). Thus 4-fold increase of the HAI titer was obtained.

Performing the assay of the T-cell response as pg/ml of INF-γ after NP stimulation, revealed that the negatively charged adjuvant enhanced the response 7-fold over the non-adjuvanted formulation.

### Example 3

A negatively charged formulation with oleic acid and mono-olein having a concentration of 4% was mixed at a 1+1 ratio (v/v) with a suspension of inactivated influenza virus particles (strain H1N1/Brisbane). The mixture was subsequently administered intranasally to humans in a 150 μl volume as drops into each nostril. The dose of the influenza virus particles was the equivalent of 5, 15 or 30 μg of hemeagglutinin (HA), combined with a concentration of 0.5, 1 or 2% of the negatively charged adjuvant formulation in various combinations in different groups. A total of 104 human subjects were immunized on three occasions, separated with three weeks with formulations containing both antigen and adjuvant. A total of 120 subjects received adjuvant formulations intranasally, including a reference formulation containing only adjuvant and no antigen. Three weeks after the last immunization blood samples were analyzed for immunological responses.

The results revealed that:

No serious adverse events related to the vaccine were seen in any of these human subjects. No case of Bell's palsy was reported among any of the subjects receiving the adjuvant. Adverse events reported included a slight transient discomfort intranasally, which disappeared within two hours after administration of the formulations. In Table 1, the reported Adverse Events, AE, are presented from the group receiving 15 μg antigen and 2% negatively charged adjuvant.

TABLE 1
Reported adverse events, AE, from the group receiving 15 μg
antigen and 2% negatively charged adjuvant in the clinical study
on humans.

Negatively
charged formulation
(3 doses, n = 18)

| Preferred term | Total AE | Related AE |
|---|---|---|
| Influenza | 1 | — |
| Nasopharyngitis | 3 | 1 |
| Pain in extremity | 1 | — |
| Dizziness | 1 | — |
| Tremor | 1 | — |
| Cough | 1 | 1 |
| Nasal congestion | 1 | 1 |
| Oropharyngeal pain | 1 | 1 |
| Throat irritation | 3 | 3 |
| Upper airway obstruction | 3 | 2 |
| Erythema | 1 | 1 |
| Pruritus | 1 | 1 |
| Total | 18 | 11 |

In order to get approval of a seasonal influenza vaccine, at least one of the following criteria has to be fulfilled (as decided by European Medical Agency, EMA). These include:

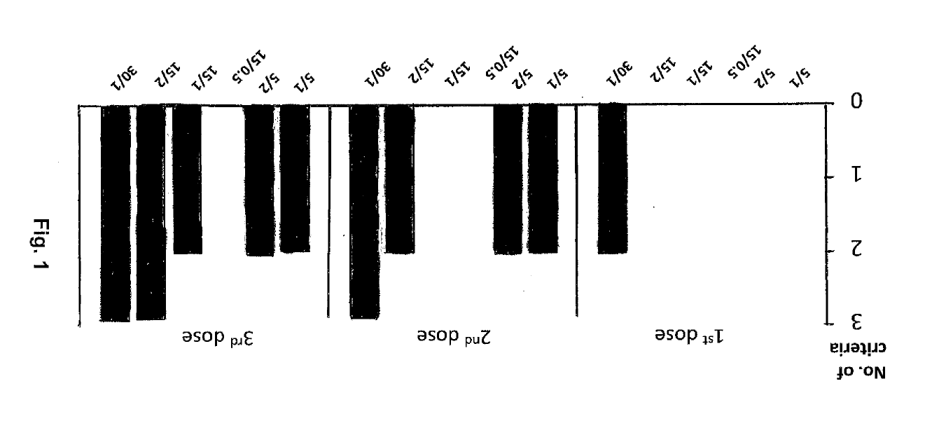

Seroconversion: Proportion of subjects achieving a significant increase in HAI, i.e. at least a 4-fold increase in titer (requirement >40%)
GMT: Geometric mean fold increase in HAI titers (requirement >2.5)
Seroprotection: Proportion of subjects achieving an HAI titer 40 (requirement >70%) As can be seen in FIG. 1, these criteria are fulfilled for one group of subjects after one administration, by 5 groups after 2 administration and by all groups after 3 administrations.

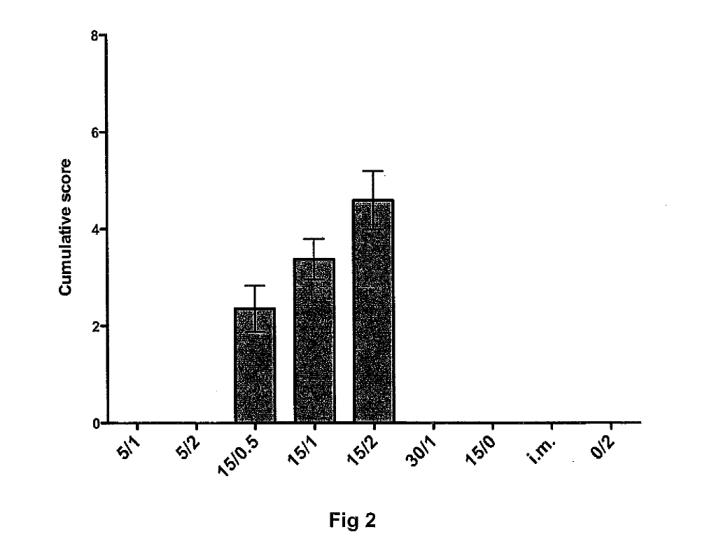

In order to assess to over-all immunogenicity of the formulations tested on the human subjects, a combination score was calculated. For example, when a subject obtained at least a 4-fold increase in a titre, he was given a score of 1 (one). The scores were then determined for the following assays: HAI, Nasal IgA against H1N1/Brisbane, Nasal IgA against H1N1/California, Nasal IgA against H₃N₂/Brisbane, Nasal IgA against H1N1/California and IFN-γ. A subject could thus obtain a maximum of 8 points. As can be seen in FIG. 2, a dose-response with regard to the concentration of the negatively charged adjuvant was obtained.

### Example 4

### Investigations of Adverse Events when Using Adjuvants According to the Invention and Diphtheria Toxoid (DT)

The study had a randomized, double blind, parallel group design. A total of 40 healthy volunteers were included and randomized to 1 of 4 treatment groups. The first group received 1% of Adjuvant B)/DT (diphtheria toxoid) which was administered with a standard applicator. Once the safety of the first group had been established, the following two groups were treated with either 4% Adjuvant B/DT or DT alone as reference, both administered with a standard applicator. In the fourth group, 4% Adjuvant B/DT was administered with a nasal applicator. Eligible subjects were immunized via the nasal mucosa on day 1. Fourteen days later the anti-diphtheria immune response to the vaccine was evaluated. Adverse events were assessed throughout the study period.

Adjuvant B/DT vaccine: 1% or 4% adjuvant B and 75 Lf/ml DT, 2×100 μl was administered via standard or nasal aerosol spray.

Thirty-eight (38) out of the 40 subjects reported a total of 107 Adverse Events (AEs). The most common type of adverse events were all mild and related to local irritation of the nasal mucosa, manifested as a stinging feeling in nose, running nose, sneezing, stuffed nose, epistaxis and tenderness in nasal cavity. No deaths, serious adverse events or discontinuations of investigational product due to adverse events were reported during the study.

### 4.1 Display and Analysis of Adverse Events

In total, 38 out of the 40 included subjects reported 107 events. Two of these events, artroscopi in Subject No. 117 and migraine in Subject No. 106, were reported as severe, 12 were reported as moderate and 93 as mild. The number of events reported per subject ranged from 1 to 8, with an average of 2.8 events. Most part of the reported AEs (63 out of 107) had a causal relationship with treatment; e.g. possible, probable or definite relationship. The two subjects who did not report any AE were included in the reference treatment group and the 1% adjuvant B/DT treatment group. All EAs are summarized by relationship to treatment in Table 6.

Nine out of the 107 events led to the administration of concomitant medication and all subjects recovered without sequelae. The most common AEs were stinging feeling in the nose (26 occasions) and headache (15 occasions). Table 2 to Table 5 display number of AEs by relationship to treatment after administration of DT alone, 1% Adjuvant B/DT, 4% Adjuvant B/DT with the standard applicator and after administration of 4% adjuvant B/DT with the nasal applicator, respectively.

TABLE 2
Adverse event by relationship to treatment after
administration of DT alone (reference).

| | Relationship^{a} to treatment | | | | | |
|---|---|---|---|---|---|---|
| Adverse event | 1 | 2 | 3 | 4 | 5 | TOTAL |
| abdominal pain | | | | | 1 | 1 |
| coughing | | | | 1 | | 1 |
| dizziness | | 1 | | | | 1 |
| dry skin in face | | | | | 1 | 1 |
| dysmenorrhea | | | | 1 | | 1 |
| headache | | | 3 | 1 | | 4 |
| intermenstrual bleeding | | | | 1 | | 1 |
| migraine | | | 1 | | | 1 |
| nausea | | | | 1 | | 1 |
| rhinitis | | | 1 | 2 | | 3 |
| running nose | | | 1 | | | 1 |
| sneezing | | | 1 | | | 1 |
| stinging feeling in nose | 1 | | | | | 1 |
| stuffed nose | | | 1 | 1 | | 2 |
| TOTAL | 1 | 1 | 8 | 8 | 2 | 20 |

^{a}1 = definite, 2 = probable, 3 = possible 4 = unlikely, 5 = no connection

TABLE 3
Adverse event by relationship to treatment after administration of
1% adjuvant B/DT.

| | Relationship^{a} to treatment | | | | |
|---|---|---|---|---|---|
| Adverse event | 1 | 3 | 4 | 5 | TOTAL |
| abdominal pain | | | | 1 | 1 |
| backpain | | | | 1 | 1 |
| dizziness | | 2 | | | 2 |
| falling and hit her head | | | | 1 | 1 |
| headache | | | | 7 | 7 |
| nausea | | | 1 | 1 | 2 |
| numbness, forehead and throat | | 1 | | | 1 |
| pharyngitis | | | | 1 | 1 |
| post commotio | | | | 1 | 1 |
| rhinitis | | | | 2 | 2 |
| running nose | 1 | | | | 1 |
| sneezing | 1 | | | | 1 |
| somnolence | | 1 | 2 | | 3 |
| stinging feeling in nose | 5 | | | | 5 |
| stuffed nose | 2 | | | | 2 |
| swollen eyelids | | | | 1 | 1 |
| TOTAL | 9 | 4 | 3 | 16 | 32 |

^{a}1 = definite, 2 = probable, 3 = possible 4 = unlikely, 5 = no connection

TABLE 4
Adverse event by relationship to treatment after
administration of 4% adjuvant B/DT.

| | Relationship^{a} to treatment | | | | | |
|---|---|---|---|---|---|---|
| Adverse event | 1 | 2 | 3 | 4 | 5 | TOTAL |
| artroscopi in knee | | | | | 1 | 1 |
| backpain | | | | | 1 | 1 |
| commedon in right nostril | | | 1 | | | 1 |
| dizziness | | 1 | 1 | | | 2 |
| epitaxis | 2 | | | | | 2 |
| headache | | | | 4 | | 4 |
| insomnia | | | | | 1 | 1 |
| migraine | | | 1 | | | 1 |
| muscle contusion | | | | | 1 | 1 |
| pharyngitis | | | | 1 | 1 | 2 |
| running nose | 3 | | | | | 3 |
| sneezed ten times | 1 | | 1 | | | 2 |
| somnolence | | | 1 | | | 1 |
| stinging feeling in nose | 10 | | | | | 10 |
| stuffed nose | | | | 2 | | 2 |
| tear filled eyes | 1 | | | | | 1 |
| toothache | | | | | 1 | 1 |
| TOTAL | 17 | 1 | 5 | 7 | 6 | 36 |

^{a}1 = definite, 2 = probable, 3 = possible 4 = unlikely, 5 = no connection

TABLE 5
Adverse event by relationship to treatment after administration of
4% adjuvant B/DT using a nose applicator.

Relationship^{a} to
treatment

| Adverse event | 1 | 3 | 5 | TOTAL |
|---|---|---|---|---|
| dyspnea | | 1 | | 1 |
| muscular pain, lifting furnitures | | | 1 | 1 |
| rhinitis | | | 1 | 1 |
| running nose | 3 | | | 3 |
| somnolence | | 1 | | 1 |
| stinging feeling in nose | 10 | | | 10 |
| stuffed nose | | 1 | | 1 |
| tenderness in nasal cavity | 1 | | | 1 |
| TOTAL | 14 | 3 | 2 | 19 |

^{a}1 = definite, 2 = probable, 3 = possible 4 = unlikely, 5 = no connection

No deaths, other serious adverse events or discontinuations due to adverse events occurred during the study. Moreover, no observations of Bell's Palsy or indications thereto was observed.

TABLE 6
Adverse events by relationship to treatment, all treatments

| | Relationship^{a} to treatment | | | | | |
|---|---|---|---|---|---|---|
| Adverse event | 1 | 2 | 3 | 4 | 5 | TOTAL |
| abdominal pain | | | | | 2 | 2 |
| artroscopi in knee | | | | | 1 | 1 |
| backpain | | | | | 2 | 2 |
| commedon in right nostril | | | 1 | | | 1 |
| coughing | | | | 1 | | 1 |
| dizziness | | 2 | 3 | | | 5 |
| dry skin in face | | | | | 1 | 1 |
| dysmenorrhea | | | | 1 | | 1 |
| dyspnea | | | 1 | | | 1 |
| epitaxis | 2 | | | | | 2 |
| falling and hit her head | | | | | 1 | 1 |
| headache | | | 3 | 5 | 7 | 15 |
| insomnia | | | | | 1 | 1 |
| intermenstruel bleeding | | | | 1 | | 1 |
| migraine | | | 2 | | | 2 |
| muscle contusion | | | | | 1 | 1 |
| muscular pain, lifting furnitures | | | | | 1 | 1 |
| nausea | | | | 2 | 1 | 3 |
| numbness, forehead and throat | | | 1 | | | 1 |
| pharyngitis | | | | 1 | 2 | 3 |
| post commotio | | | | | 1 | 1 |
| rhinitis | | | 1 | 2 | 3 | 6 |
| running nose | 7 | | 1 | | | 8 |
| sneezed ten times | 1 | | 1 | | | 2 |
| sneezing | 1 | | 1 | | | 2 |
| somnolence | | | 3 | 2 | | 5 |
| stinging feeling in nose | 26 | | | | | 26 |
| stuffed nose | 2 | | 2 | 3 | | 7 |
| swollen eyelids | | | | | 1 | 1 |
| tear filled eyes | 1 | | | | | 1 |
| tenderness in nasal cavity | 1 | | | | | 1 |
| toothache | | | | | 1 | 1 |
| TOTAL | 41 | 2 | 20 | 18 | 26 | 107 |

^{a}1 = definite, 2 = probable, 3 = possible 4 = unlikely, 5 = no connection

### Example 5

The test items were different concentrations (0.5, and 2%) of Adjuvant B and 2% Adjuvant B with an antigen concentration of 0.1 μg/μl. As control item the vehicle, 0.1 M TRIS buffer, was used. The study was performed on 60 SD rats of both genders (30 males and 30 females). The animals were divided into five groups (6 female and 6 male rats/group). All animals were treated by nasal administration with vehicle, adjuvant or adjuvant+virus antigen, in a volume of 50 μl (about 25 μl into each nostril) by a catheter. After administration the animals were kept anaesthetized for 5 minutes to allow absorption of fluid and to minimize the risk for outflow of the solution from the nasal cavity. The administrations were performed 4 times 9-10 days in between. The animals were treated as follows:

Group 1: Vehicle; Group 2: Low dose (0.5%) adjuvant; Group 3: High dose (2%) adjuvant; Group 4: High dose (2%) adjuvant+virus antigen serotype H1N1 (5 μg); Group 5: High dose (2%) adjuvant+virus antigen serotype H1N1 (5 μg), recovery The weight of the animals was recorded on arrival, after conditioning, then 3 times weekly, and finally at termination of study. No statistically significant differences in weights between treatment groups were found. The animals were checked daily for change in food intake, activity etc as signs of change in general health status. No abnormalities in health status were recorded. One day after the last administration the first three animals in the groups 1, 2, 3 and 4, and two days after the last administration the remaining animals in groups 1, 2, 3 and 4, were anaesthetised and blood and serum was obtained for haematology, clinical chemistry and immunology analyses. One week after the last treatment the animals in group 5 were subjected to blood samplings as of above. There were no statistically significant differences between groups with regards to the haematological parameters tested. Clinical chemistry showed no statistically significant differences between groups of females. Among males, LDH for vehicle treated animals was statistically significantly higher than animals treated with high dose adjuvant+virus antigen. This observation has probably no practical meaning since the observed LDH for the vehicle treated males is higher than normally observed for Sprague-Dawley rats.

Immunological analyses of serum showed that animals in all groups, also animals in the groups that were not intentionally treated with virus antigen, had antibodies against Influenza A (H1N1/PR8). However, 12/12 animals in group 5 and 9/12 animals in group 4 were seropositive which shows that the antigen was effective in combination with the 2% adjuvant to stimulate antibody production against Influenza A. The presence of antibodies in the groups of animals not intentionally treated with Influenza A indicates that these animals at some point had been exposed to Influenza A which is an observation not uncommon in animals which have not been bred in an isolator-cage environment. One day after the last administration the first three animals in groups 1, 2, 3 and 4, and two days after the last administration the remaining animals in groups 1, 2, 3 and 4, were euthanized and lungs, heart, liver, spleen, pancreas, kidneys, gonads, ileum, mesenteric lymph nodes, axillary lymph nodes, mandibular lymph nodes, thymus, bone marrow, and skulls were dissected. One week after the last treatment all animals in group 5 were subjected to organ sampling as of above. After fixation and paraffin embedding the tissues were sectioned and subsequently examined for histopathological changes. Histopathology showed lesions only in the nasal mucosa, all other dissected organs were normal. Since the adjuvant and virus antigen was administered into the nasal cavity and caused an immunological response, one would expect to find signs of an inflammatory reaction in the nasal mucosa. Lesions were, however, also found in the vehicle treated animals which makes interpretation of the histopathological findings difficult since the vehicle would not cause such effects. The fact that the animals used in the present study seemingly had been exposed to Influenza A may be one cause of the observed lesions but other infectious agents cannot be excluded. It seems unlikely that the adjuvant per se caused lesions since 0/6 males had lesions in the group of animals treated with the high dose of adjuvant. Thus, the frequency of lesions observed in the group of male animals treated with the low dose of adjuvant may represent the high normal frequency of nasal lesions in this group of animals. Therefore, it may be concluded that the frequency of lesions in the groups of animals treated with adjuvant and virus antigen was not higher than in any other group of animals. In fact, Chi-Square analysis showed no statistically significant difference in the frequency of lesions among groups of treatment. It is concluded that the H1N1 antigen used in this study provoked an immunological response which stimulated antibody production against H1N1 virus. The severity and frequency of lesions in adjuvant or adjuvant+virus antigen treated animals were similar to frequency of lesions found in vehicle treated animals. This indicates that the adjuvant or adjuvant+virus antigen did not produce mucosal lesions. Health records haematology, clinical chemistry and histopathology analyses indicate that the vaccine, after repeated administration, did not cause general toxic reactions in rats.

### Summary of Histopathological Findings Regarding Lesions in Nasal Mucosa

Table 7 shows a summary of lesions observed in the nasal mucosa at four sectioned levels (L1-L4). Level L4b represents the level in which also the olfactory bulb (OB) was present. An X indicates that a lesion was found, W/OAR (With Out Any Remarks) indicates that no remarks were noted about the status of the organ. The lesions were in the form of blood in the nasal cavity, erosions of the epithelium in the nasal mucosa, oedema, or inflammation. The column denoted “Lesion” was used to statistically analyse the frequency of lesions between groups.

TABLE 7

| ID | Gender | Group | Lesion | L1 | L2 | L3 | L4 | L4b | OB |
|---|---|---|---|---|---|---|---|---|---|
| 585 | M | 1 | 1 | X | X | X | X | X | W/OAR |
| 586 | M | 1 | 0 | W/OAR | W/OAR | W/OAR | W/OAR | W/OAR | W/OAR |
| 587 | M | 1 | 0 | W/OAR | W/OAR | W/OAR | W/OAR | W/OAR | W/OAR |
| 588 | M | 1 | 0 | W/OAR | W/OAR | W/OAR | W/OAR | W/OAR | W/OAR |
| 589 | M | 1 | 1 | W/OAR | W/OAR | X | X | X | W/OAR |
| 590 | M | 1 | 0 | W/OAR | W/OAR | W/OAR | W/OAR | W/OAR | W/OAR |
| 615 | F | 1 | 0 | W/OAR | W/OAR | W/OAR | W/OAR | W/OAR | W/OAR |
| 616 | F | 1 | 0 | W/OAR | W/OAR | W/OAR | W/OAR | W/OAR | W/OAR |
| 617 | F | 1 | 1 | W/OAR | W/OAR | W/OAR | X | X | W/OAR |
| 618 | F | 1 | 0 | W/OAR | W/OAR | W/OAR | W/OAR | W/OAR | W/OAR |
| 619 | F | 1 | 0 | W/OAR | W/OAR | W/OAR | W/OAR | W/OAR | W/OAR |
| 620 | F | 1 | 0 | W/OAR | W/OAR | W/OAR | W/OAR | W/OAR | W/OAR |
| 591 | M | 2 | 0 | W/OAR | W/OAR | W/OAR | W/OAR | W/OAR | W/OAR |
| 592 | M | 2 | 1 | W/OAR | W/OAR | W/OAR | X | W/OAR | W/OAR |
| 593 | M | 2 | 1 | W/OAR | W/OAR | X | X | X | W/OAR |
| 594 | M | 2 | 0 | W/OAR | W/OAR | W/OAR | W/OAR | W/OAR | W/OAR |
| 595 | M | 2 | 1 | W/OAR | W/OAR | W/OAR | X | W/OAR | W/OAR |
| 596 | M | 2 | 1 | X | W/OAR | X | X | X | W/OAR |
| 621 | F | 2 | 0 | W/OAR | W/OAR | W/OAR | W/OAR | W/OAR | W/OAR |
| 622 | F | 2 | 0 | W/OAR | W/OAR | W/OAR | W/OAR | W/OAR | W/OAR |
| 623 | F | 2 | 0 | W/OAR | W/OAR | W/OAR | W/OAR | W/OAR | W/OAR |
| 624 | F | 2 | 0 | W/OAR | W/OAR | W/OAR | W/OAR | W/OAR | W/OAR |
| 625 | F | 2 | 0 | W/OAR | W/OAR | W/OAR | W/OAR | W/OAR | Missing |
| 626 | F | 2 | 1 | W/OAR | W/OAR | W/OAR | W/OAR | X | W/OAR |
| 597 | M | 3 | 0 | W/OAR | W/OAR | W/OAR | W/OAR | W/OAR | W/OAR |
| 598 | M | 3 | 0 | W/OAR | W/OAR | W/OAR | W/OAR | W/OAR | W/OAR |
| 599 | M | 3 | 0 | W/OAR | W/OAR | W/OAR | W/OAR | W/OAR | W/OAR |
| 600 | M | 3 | 0 | W/OAR | W/OAR | W/OAR | W/OAR | W/OAR | W/OAR |
| 601 | M | 3 | 0 | W/OAR | W/OAR | W/OAR | W/OAR | W/OAR | W/OAR |
| 602 | M | 3 | 0 | W/OAR | W/OAR | W/OAR | W/OAR | W/OAR | W/OAR |
| 627 | F | 3 | 0 | W/OAR | W/OAR | W/OAR | W/OAR | W/OAR | W/OAR |
| 628 | F | 3 | 1 | W/OAR | W/OAR | W/OAR | X | W/OAR | W/OAR |
| 629 | F | 3 | 1 | W/OAR | W/OAR | X | X | Missing | Missing |
| 630 | F | 3 | 1 | W/OAR | W/OAR | W/OAR | X | X | W/OAR |
| 631 | F | 3 | 0 | W/OAR | W/OAR | W/OAR | W/OAR | W/OAR | W/OAR |
| 632 | F | 3 | 0 | W/OAR | W/OAR | W/OAR | W/OAR | W/OAR | W/OAR |
| 603 | M | 4 | 0 | W/OAR | W/OAR | W/OAR | W/OAR | W/OAR | W/OAR |
| 604 | M | 4 | 1 | W/OAR | W/OAR | X | W/OAR | W/OAR | W/OAR |
| 605 | M | 4 | 1 | W/OAR | W/OAR | X | X | W/OAR | W/OAR |
| 606 | M | 4 | 1 | W/OAR | W/OAR | X | X | X | W/OAR |
| 607 | M | 4 | 1 | W/OAR | W/OAR | X | X | W/OAR | W/OAR |
| 608 | M | 4 | 1 | X | W/OAR | X | X | W/OAR | W/OAR |
| 633 | F | 4 | 0 | W/OAR | W/OAR | W/OAR | W/OAR | W/OAR | W/OAR |
| 634 | F | 4 | 0 | W/OAR | W/OAR | W/OAR | W/OAR | W/OAR | W/OAR |
| 635 | F | 4 | 0 | W/OAR | W/OAR | W/OAR | W/OAR | W/OAR | W/OAR |
| 636 | F | 4 | 0 | W/OAR | W/OAR | W/OAR | W/OAR | W/OAR | W/OAR |
| 637 | F | 4 | 1 | W/OAR | W/OAR | X | X | W/OAR | W/OAR |
| 638 | F | 4 | 0 | W/OAR | W/OAR | W/OAR | W/OAR | W/OAR | W/OAR |
| 609 | M | 5 | 0 | W/OAR | W/OAR | W/OAR | W/OAR | W/OAR | W/OAR |
| 610 | M | 5 | 0 | W/OAR | W/OAR | W/OAR | W/OAR | W/OAR | W/OAR |
| 611 | M | 5 | 1 | X | W/OAR | X | X | X | W/OAR |
| 612 | M | 5 | 1 | W/OAR | W/OAR | W/OAR | X | W/OAR | W/OAR |
| 613 | M | 5 | 1 | W/OAR | W/OAR | X | X | X | W/OAR |
| 614 | M | 5 | 1 | W/OAR | W/OAR | W/OAR | X | X | W/OAR |
| 639 | F | 5 | 1 | W/OAR | W/OAR | X | X | W/OAR | W/OAR |
| 640 | F | 5 | 0 | W/OAR | W/OAR | W/OAR | W/OAR | W/OAR | W/OAR |
| 641 | F | 5 | 0 | W/OAR | W/OAR | W/OAR | W/OAR | W/OAR | W/OAR |
| 642 | F | 5 | 1 | W/OAR | W/OAR | X | X | X | W/OAR |
| 643 | F | 5 | 1 | W/OAR | W/OAR | W/OAR | X | X | W/OAR |
| 644 | F | 5 | 1 | W/OAR | W/OAR | X | X | X | W/OAR |

### 5.1 Frequency of the Presence of Lesions Between Groups of Treatment

Tables 8a-c shows number of animals in each group of treatment observed to have lesions in the nasal mucosa (lesion=1) and animals without lesions (lesion=0).

### Tables 8a-c

| | | Group | | | | | |
|---|---|---|---|---|---|---|---|
| | | | | | High | | |
| | | | | High | dose | High dose | |
| | | | | dose | adj + | adj + virus | |
| | | | Low dose | adjuv- | virus | antigen, | |
| Females | | Vehicle | adjuvant | ant | antigen | recovery | Total |
| Lesion | 0 | 5 | 5 | 3 | 5 | 2 | 20 |
| | 1 | 1 | 1 | 3 | 1 | 4 | 10 |
| Total | | 6 | 6 | 6 | 6 | 6 | 30 |

| | | Group | | | | | |
|---|---|---|---|---|---|---|---|
| | | | | | High | | |
| | | | Low | | dose | High dose | |
| | | | dose | | adj + | adj + virus | |
| | | | adju- | High dose | virus | antigen, | |
| Males | | Vehicle | vant | adjuvant | antigen | recovery | Total |
| Lesion | 0 | 4 | 2 | 6 | 1 | 2 | 15 |
| | 1 | 2 | 4 | 0 | 5 | 4 | 15 |
| Total | | 6 | 6 | 6 | 6 | 6 | 30 |

| Chi-Square Tests | Value | df | Asymp. Sig. (2-sided) |
|---|---|---|---|
| Pearson Chi-Square | 6.17 | 4 | 0.187 |
| Likelihood Ratio | 6.30 | 4 | 0.178 |
| Linear-by-Linear Association | 4.08 | 1 | 0.043 |

### Example 6

The test items will be different concentrations (0.5, and 2%) of Adjuvant B, antigen (5 μg) and 0.5 or 2% adjuvant with an antigen concentration of 0.1 μg/μl. Since the test items will be administered in a total volume of 50 μl to each rat the H1N1 containing test item will be designated as 0.5 or 2% Adjuvant+5 μg antigen.

| | Volume | Requested | |
|---|---|---|---|
| | needed | volume | |
| Test item | (μl) | (μl) | Dispensed in |
| Adjuvant, 0.5% | 2400 | 3200 | 4 vials (800 μl in each) |
| (Batch nr: 95002- | | | |
| 0810-6) | | | |
| Adjuvant, 2% (Batch | 2400 | 3200 | 4 vials (800 μl in each) |
| nr: 95002-0810-17) | | | |
| Antigen, 5 μg (Batch | 2400 | 3200 | 4 vials (800 μl in each) |
| nr: 95002-0810-18) | | | |
| Adjuvant, 0.5% + | 2400 | 3200 | 4 vials (800 μl in each) |
| 5 μg antigen (Batch | | | |
| nr: 95002-0810-19) | | | |
| Adjuvant, 2% + 5 μg | 4800 | 6400 | 4 vials (1600 μl in each) |
| antigen (Batch nr: | | | |
| 95002-0810-20 | | | |

### Control Item

Vehicle, 0.1M TRIS buffer

| | | Requested | |
|---|---|---|---|
| | Volume needed | volume | |
| Control item | (μl) | (μl) | Dispensed in |
| Vehicle (Batch nr: | 2400 | 3200 | 4 vials (800 μl in each) |
| 95002-0810-12) | | | |

### Materials and Methods

### Animals:

The study will be performed using 84 SD rats of both genders (42 males and 42 females), weighing about 200 g at arrival, (ID 2244-8 to 2285-8 males and ID 2287-8 to 2328-8 females). The animals will be obtained from Scanbur BK, Sollentuna, Sweden. The animals will be kept in M4 cages (3 in each). They will be provided with free access to water and a commercial diet, R34, from Lactamin AB, Sweden. All animals will be acclimatized for a minimum of 7 days prior to the commencement of the experiment. The experiment is approved by the regional animal experimental ethics committee in Uppsala (C25/7). Rats are chosen because they are easy to handle in this kind of studies.

### Administration of Test and Control Items:

The animals will be divided into seven groups (6 female and 6 male rats/group). All animals will be treated by nasal administration (under light Isoflurane anaesthesia) with vehicle, adjuvant, antigen or adjuvant+virus antigen, in a volume of 50 μl (approximately 25 μl into each nostril) by a pipette. After administration the animals will be kept anaesthetized for a few minutes to allow absorption of fluid and to minimize the risk for outflow of the solution from the nasal cavity. The administrations will be performed 4 times 14 days in between. The animals will be treated as follows:

### Group 1: Vehicle

Group 2: Low dose (0.5%) adjuvant
Group 3: High dose (2%) adjuvant
Group 4: Virus antigen (alone) serotype H1N1 (5 μg)
Group 5: Low dose adjuvant 0.5%+virus antigen (5 μg)
Group 6: High dose adjuvant 2%+virus antigen (5 μg)
Group 7: High dose adjuvant 2%+virus antigen (5 μg)

### Technical Notes

The tissues were subdivided into a number of cassettes labeled as follows:

K1: scull, level 1
K2: scull, level 2
K3: scull, level 3
K4: scull, level 4.

Unless otherwise specified in the table, there is one slide per tissue block. Slides with new sections (i e tissue blocks subjected to re-sectioning) are labeled n.s. (“new section”). Slides labeled “niv8 . . . ” (level) reflect that sections are taken from more than one level.

Group 1:

| Slide labeling | Check | Analysis |
|---|---|---|
| VP08-35 | ✓ | Micromorphology without remark. |
| MM08043 | | |
| 2244:K1 | | |
| VP08-35 | ✓ | Micromorphology without remark. |
| MM08043 | | |
| 2244:K2 | | |
| VP08-35 | ✓ | Micromorphology without remark. |
| MM08043 | | |
| 2244:K3 | | |
| VP08-35 | ✓ | Micromorphology without remark. |
| MM08043 | | |
| 2244:K4 | | |
| VP08-35 | ✓ | Micromorphology without remark. |
| MM08043 | | |
| 2245:K1 | | |
| VP08-35 | ✓ | Micromorphology without remark. |
| MM08043 | | |
| 2245:K2 | | |
| VP08-35 | ✓ | Micromorphology without remark. |
| MM08043 | | |
| 2245:K3 | | |
| VP08-35 | ✓ | Micromorphology without remark. |
| MM08043 | | |
| 2245:K4 | | |
| (2 slides) | | |
| VP08-35 | ✓ | Micromorphology without remark. |
| MM08043 | | |
| 2246:K1 | | |
| VP08-35 | ✓ | Micromorphology without remark. |
| MM08043 | | |
| 2246:K2 | | |
| VP08-35 | ✓ | Micromorphology without remark. |
| MM08043 | | |
| 2246:K3 | | |
| VP08-35 | ✓ | Micromorphology without remark. |
| MM08043 | | |
| 2246:K4 | | |
| VP08-35 | ✓ | Micromorphology without remark. |
| MM08043 | | |
| 2247:K1 | | |
| VP08-35 | ✓ | Micromorphology without remark. |
| MM08043 | | |
| 2247:K2 | | |
| VP08-35 | ✓ | Serous secretions containing loosened necrotic epithelial cells and |
| MM08043 | | small amount of blood can be seen in the posterior part of the |
| 2247:K3 | | nasal cavity. |
| VP08-35 | ✓ | Micromorphology without remark. |
| MM08043 | | |
| 2247:K4 | | |
| VP08-35 | ✓ | Micromorphology without remark. |
| MM08043 | | |
| 2248:K1 | | |
| VP08-35 | ✓ | Micromorphology without remark. |
| MM08043 | | |
| 2248:K2 | | |
| VP08-35 | ✓ | Fresh hemorrhage in the nasal cavity. |
| MM08043 | | |
| 2248:K3 | | |
| VP08-35 | ✓ | Micromorphology without remark. |
| MM08043 | | |
| 2248:K4 | | |
| VP08-35 | ✓ | Micromorphology without remark. |
| MM08043 | | |
| 2249:K1 | | |
| VP08-35 | ✓ | Micromorphology without remark. |
| MM08043 | | |
| 2249:K2 | | |
| VP08-35 | ✓ | Fresh hemorrhage in the nasal cavity and nasopharynx. |
| MM08043 | | |
| 2249:K3 | | |
| VP08-35 | ✓ | Micromorphology without remark. |
| MM08043 | | |
| 2249:K4 | | |
| VP08-35 | ✓ | Micromorphology without remark. |
| MM08043 | | |
| 2287:K1 | | |
| (2 slides) | | |
| VP08-35 | ✓ | Micromorphology without remark. |
| MM08043 | | |
| 2287:K2 | | |
| VP08-35 | ✓ | The olfactory epithelium shows oedema and detaching of cells into |
| MM08043 | | the nasal cavity which also contains fresh bleeding and serous |
| 2287:K3 | | fluid. Fresh bleeding in nasopharynx. |
| VP08-35 | ✓ | Micromorphology without remark. |
| MM08043 | | |
| 2287:K4 | | |
| VP08-35 | ✓ | Micromorphology without remark. |
| MM08043 | | |
| 2288:K1 | | |
| (3 slides) | | |
| VP08-35 | ✓ | Micromorphology without remark. |
| MM08043 | | |
| 2288:K2 | | |
| VP08-35 | ✓ | Fresh hemorrhage in the nasal cavity. Oedema in the olfactory |
| MM08043 | | epithelium with detachment of the epithelial cells. |
| 2288:K3 | | |
| VP08-35 | ✓ | Micromorphology without remark. |
| MM08043 | | |
| 2288:K4 | | |
| (2 slides) | | |
| VP08-35 | ✓ | Micromorphology without remark. |
| MM08043 | | |
| 2289:K1 | | |
| (2 slides) | | |
| VP08-35 | ✓ | Micromorphology without remark. |
| MM08043 | | |
| 2289:K2 | | |
| VP08-35 | ✓ | Fresh hemorrhage is the nasal cavity. Oedema in the olfactory |
| MM08043 | | epithelium with detachment of the epithelial cells. Fresh |
| 2289:K3 | | hemorrhage in nasopharynx. |
| VP08-35 | ✓ | Micromorphology without remark. |
| MM08043 | | |
| 2289:K4 | | |
| VP08-35 | ✓ | Micromorphology without remark. |
| MM08043 | | |
| 2290:K1 | | |
| (3 slides) | | |
| VP08-35 | ✓ | Micromorphology without remark. |
| MM08043 | | |
| 2290:K2 | | |
| VP08-35 | ✓ | Massive oedema in the olfactory epithelium with detachment of the |
| MM08043 | | epithelial cells. No bleeding in the nasal cavity. |
| 2290:K3 | | |
| VP08-35 | ✓ | Micromorphology without remark. |
| MM08043 | | |
| 2290:K4 | | |
| VP08-35 | ✓ | Micromorphology without remark. |
| MM08043 | | |
| 2291:K1 | | |
| (2 slides) | | |
| VP08-35 | ✓ | Micromorphology without remark. |
| MM08043 | | |
| 2291:K2 | | |
| VP08-35 | ✓ | Fresh hemorrhage is the nasal cavity. Oedema in the olfactory |
| MM08043 | | epithelium with detachment of the epithelial cells. |
| 2291:K3 | | |
| VP08-35 | ✓ | Micromorphology without remark. |
| MM08043 | | |
| 2291:K4 | | |
| VP08-35 | ✓ | Micromorphology without remark. |
| MM08043 | | |
| 2292:K1 | | |
| VP08-35 | ✓ | Micromorphology without remark. |
| MM08043 | | |
| 2292:K2 | | |
| VP08-35 | ✓ | Massive oedema in the olfactory epithelium with detachment of the |
| MM08043 | | epithelial cells. No bleeding in the nasal cavity. |
| 2292:K3 | | |
| VP08-35 | ✓ | Micromorphology without remark. |
| MM08043 | | |
| 2292:K4 | | |

Group 2

| Slide labeling | Check | Analysis |
|---|---|---|
| VP08-35 | ✓ | Micromorphology without remark. |
| MM08043 | | |
| 2250:K1 | | |
| VP08-35 | ✓ | Micromorphology without remark. |
| MM08043 | | |
| 2250:K2 | | |
| VP08-35 | ✓ | Micromorphology without remark. |
| MM08043 | | |
| 2250:K3 | | |
| VP08-35 | ✓ | Micromorphology without remark. |
| MM08043 | | |
| 2250:K4 | | |
| VP08-35 | ✓ | Micromorphology without remark. |
| MM08043 | | |
| 2251:K1 | | |
| VP08-35 | ✓ | Micromorphology without remark. |
| MM08043 | | |
| 2251:K2 | | |
| (2 slides) | | |
| VP08-35 | ✓ | Micromorphology without remark. |
| MM08043 | | |
| 2251:K3 | | |
| VP08-35 | ✓ | Micromorphology without remark. |
| MM08043 | | |
| 2251:K4 | | |
| VP08-35 | ✓ | Micromorphology without remark. |
| MM08043 | | |
| 2252:K1 | | |
| VP08-35 | ✓ | Micromorphology without remark. |
| MM08043 | | |
| 2252:K2 | | |
| VP08-35 | ✓ | Micromorphology without remark. |
| MM08043 | | |
| 2252:K3 | | |
| VP08-35 | ✓ | Micromorphology without remark. |
| MM08043 | | |
| 2252:K4 | | |
| VP08-35 | ✓ | Micromorphology without remark. |
| MM08043 | | |
| 2253:K1 | | |
| VP08-35 | ✓ | Micromorphology without remark. |
| MM08043 | | |
| 2253:K2 | | |
| VP08-35 | ✓ | Micromorphology without remark. |
| MM08043 | | |
| 2253:K3 | | |
| VP08-35 | ✓ | Micromorphology without remark. |
| MM08043 | | |
| 2253:K4 | | |
| VP08-35 | ✓ | Micromorphology without remark. |
| MM08043 | | |
| 2254:K1 | | |
| VP08-35 | ✓ | Micromorphology without remark. |
| MM08043 | | |
| 2254:K2 | | |
| VP08-35 | ✓ | Micromorphology without remark. |
| MM08043 | | |
| 2254:K3 | | |
| VP08-35 | ✓ | Micromorphology without remark. |
| MM08043 | | |
| 2254:K4 | | |
| VP08-35 | ✓ | Micromorphology without remark. |
| MM08043 | | |
| 2255:K1 | | |
| VP08-35 | ✓ | Micromorphology without remark. |
| MM08043 | | |
| 2255:K2 | | |
| VP08-35 | ✓ | Focally oedema in the olfactory |
| MM08043 | | epithelium with detachment of the |
| 2255:K3 | | epithelial cells. No hemorrhage can be seen. |
| VP08-35 | ✓ | Micromorphology without remark. |
| MM08043 | | |
| 2255:K4 | | |
| (2 slides) | | |
| VP08-35 | ✓ | Micromorphology without remark. |
| MM08043 | | |
| 2293:K1 | | |
| VP08-35 | ✓ | Micromorphology without remark. |
| MM08043 | | |
| 2293:K2 | | |
| VP08-35 | ✓ | Micromorphology without remark. |
| MM08043 | | |
| 2293:K3 | | |
| VP08-35 | ✓ | Micromorphology without remark. |
| MM08043 | | |
| 2293:K4 | | |
| VP08-35 | ✓ | Micromorphology without remark. |
| MM08043 | | |
| 2294:K1 | | |
| VP08-35 | ✓ | Micromorphology without remark. |
| MM08043 | | |
| 2294:K2 | | |
| VP08-35 | ✓ | Micromorphology without remark. |
| MM08043 | | |
| 2294:K3 | | |
| VP08-35 | ✓ | Micromorphology without remark. |
| MM08043 | | |
| 2294:K4 | | |
| VP08-35 | ✓ | Micromorphology without remark. |
| MM08043 | | |
| 2295:K1 | | |
| VP08-35 | ✓ | Micromorphology without remark. |
| MM08043 | | |
| 2295:K2 | | |
| VP08-35 | ✓ | Micromorphology without remark. |
| MM08043 | | |
| 2295:K3 | | |
| VP08-35 | ✓ | Micromorphology without remark. |
| MM08043 | | |
| 2295:K4 | | |
| VP08-35 | ✓ | Micromorphology without remark. |
| MM08043 | | |
| 2296:K1 | | |
| VP08-35 | ✓ | Micromorphology without remark. |
| MM08043 | | |
| 2296:K2 | | |
| VP08-35 | ✓ | Micromorphology without remark. |
| MM08043 | | |
| 2296:K3 | | |
| VP08-35 | ✓ | Micromorphology without remark. |
| MM08043 | | |
| 2296:K4 | | |
| VP08-35 | ✓ | Micromorphology without remark. |
| MM08043 | | |
| 2297:K1 | | |
| VP08-35 | ✓ | Micromorphology without remark. |
| MM08043 | | |
| 2297:K2 | | |
| VP08-35 | ✓ | Micromorphology without remark. |
| MM08043 | | |
| 2297:K3 | | |
| VP08-35 | ✓ | Micromorphology without remark. |
| MM08043 | | |
| 2297:K4 | | |
| VP08-35 | ✓ | Micromorphology without remark. |
| MM08043 | | |
| 2298:K1 | | |
| VP08-35 | ✓ | Micromorphology without remark. |
| MM08043 | | |
| 2298:K2 | | |
| VP08-35 | ✓ | Micromorphology without remark. |
| MM08043 | | |
| 2298:K3 | | |
| VP08-35 | ✓ | Micromorphology without remark. |
| MM08043 | | |
| 2298:K4 | | |

Group 3

| Slide labeling | Check | Analysis |
|---|---|---|
| VP08-35 | ✓ | Micromorphology without remark. |
| MM08043 | | |
| 2256:K1 | | |
| VP08-35 | ✓ | Micromorphology without remark. |
| MM08043 | | |
| 2256:K2 | | |
| VP08-35 | ✓ | Fresh hemorrhage in the nasal cavity. |
| MM08043 | | Oedema in the olfactory epithelium |
| 2256:K3 | | with detachment of the epithelial cells. |
| VP08-35 | ✓ | Micromorphology without remark. |
| MM08043 | | |
| 2256:K4 | | |
| VP08-35 | ✓ | Micromorphology without remark. |
| MM08043 | | |
| 2257:K1 | | |
| (2 slides) | | |
| VP08-35 | ✓ | Micromorphology without remark. |
| MM08043 | | |
| 2257:K2 | | |
| VP08-35 | ✓ | Micromorphology without remark. |
| MM08043 | | |
| 2257:K3 | | |
| VP08-35 | ✓ | Micromorphology without remark. |
| MM08043 | | |
| 2257:K4 | | |
| VP08-35 | ✓ | Micromorphology without remark. |
| MM08043 | | |
| 2258:K1 | | |
| VP08-35 | ✓ | Micromorphology without remark. |
| MM08043 | | |
| 2258:K2 | | |
| VP08-35 | ✓ | Micromorphology without remark. |
| MM08043 | | |
| 2258:K3 | | |
| VP08-35 | ✓ | Micromorphology without remark. |
| MM08043 | | |
| 2258:K4 | | |
| VP08-35 | ✓ | Micromorphology without remark. |
| MM08043 | | |
| 2259:K1 | | |
| VP08-35 | ✓ | Micromorphology without remark. |
| MM08043 | | |
| 2259:K2 | | |
| VP08-35 | ✓ | Micromorphology without remark. |
| MM08043 | | |
| 2259:K3 | | |
| VP08-35 | ✓ | Micromorphology without remark. |
| MM08043 | | |
| 2259:K4 | | |
| VP08-35 | ✓ | Micromorphology without remark. |
| MM08043 | | |
| 2260:K1 | | |
| (2 slides) | | |
| VP08-35 | ✓ | Micromorphology without remark. |
| MM08043 | | |
| 2260:K2 | | |
| VP08-35 | ✓ | Micromorphology without remark. |
| MM08043 | | |
| 2260:K3 | | |
| VP08-35 | ✓ | Micromorphology without remark. |
| MM08043 | | |
| 2260:K4 | | |
| VP08-35 | ✓ | Micromorphology without remark. |
| MM08043 | | |
| 2261:K1 | | |
| (2 slides) | | |
| VP08-35 | ✓ | Micromorphology without remark. |
| MM08043 | | |
| 2261:K2 | | |
| VP08-35 | ✓ | Oedema in the olfactory epithelium |
| MM08043 | | with detachment of the epithelial cells. |
| 2261:K3 | | Serous fluid in the nasal cavity. No |
| | | hemorrhage. |
| VP08-35 | ✓ | Micromorphology without remark. |
| MM08043 | | |
| 2261:K4 | | |
| VP08-35 | ✓ | Micromorphology without remark. |
| MM08043 | | |
| 2299:K1 | | |
| VP08-35 | ✓ | Micromorphology without remark. |
| MM08043 | | |
| 2299:K2 | | |
| VP08-35 | ✓ | Micromorphology without remark. |
| MM08043 | | |
| 2299:K3 | | |
| VP08-35 | ✓ | Micromorphology without remark. |
| MM08043 | | |
| 2299:K4 | | |
| VP08-35 | ✓ | Micromorphology without remark. |
| MM08043 | | |
| 2300:K1 | | |
| (2 slides) | | |
| VP08-35 | ✓ | Micromorphology without remark. |
| MM08043 | | |
| 2300:K2 | | |
| VP08-35 | ✓ | Micromorphology without remark. |
| MM08043 | | |
| 2300:K3 | | |
| VP08-35 | ✓ | Micromorphology without remark. |
| MM08043 | | |
| 2300:K4 | | |
| VP08-35 | ✓ | Micromorphology without remark. |
| MM08043 | | |
| 2301:K1 | | |
| VP08-35 | ✓ | Micromorphology without remark. |
| MM08043 | | |
| 2301:K2 | | |
| VP08-35 | ✓ | Micromorphology without remark. |
| MM08043 | | |
| 2301:K3 | | |
| VP08-35 | ✓ | Micromorphology without remark. |
| MM08043 | | |
| 2301:K4 | | |
| VP08-35 | ✓ | Micromorphology without remark. |
| MM08043 | | |
| 2302:K1 | | |
| (2 slides) | | |
| VP08-35 | ✓ | Micromorphology without remark. |
| MM08043 | | |
| 2302:K2 | | |
| VP08-35 | ✓ | Micromorphology without remark. |
| MM08043 | | |
| 2302:K3 | | |
| VP08-35 | ✓ | Micromorphology without remark. |
| MM08043 | | |
| 2302:K4 | | |
| VP08-35 | ✓ | Micromorphology without remark. |
| MM08043 | | |
| 2303:K1 | | |
| VP08-35 | ✓ | Micromorphology without remark. |
| MM08043 | | |
| 2303:K2 | | |
| VP08-35 | ✓ | Micromorphology without remark. |
| MM08043 | | |
| 2303:K3 | | |
| VP08-35 | ✓ | Micromorphology without remark. |
| MM08043 | | |
| 2303:K4 | | |
| VP08-35 | ✓ | Micromorphology without remark. |
| MM08043 | | |
| 2304:K1 | | |
| VP08-35 | ✓ | Micromorphology without remark. |
| MM08043 | | |
| 2304:K2 | | |
| VP08-35 | ✓ | Micromorphology without remark. |
| MM08043 | | |
| 2304:K3 | | |
| VP08-35 | ✓ | Micromorphology without remark. |
| MM08043 | | |
| 2304:K4 | | |

Group 4

| Slide labeling | Check | Analysis |
|---|---|---|
| VP08-35 | ✓ | Micromorphology without remark. |
| MM08043 | | |
| 2262:K1 | | |
| VP08-35 | ✓ | Micromorphology without remark. |
| MM08043 | | |
| 2262:K2 | | |
| VP08-35 | ✓ | Fresh hemorrhage in the dorsal part |
| MM08043 | | nasal cavity. The olfactory epithelium |
| 2262:K3 | | shows oedema with detachment of the |
| | | epithelial cells (most pronounced in the |
| | | lowest part of the cavity) and is covered |
| | | by a thin layer of serous fluid. |
| VP08-35 | ✓ | Micromorphology without remark. |
| MM08043 | | |
| 2262:K4 | | |
| VP08-35 | ✓ | Micromorphology without remark. |
| MM08043 | | |
| 2263:K1 | | |
| VP08-35 | ✓ | Micromorphology without remark. |
| MM08043 | | |
| 2263:K2 | | |
| (2 slides) | | |
| VP08-35 | ✓ | Micromorphology without remark. |
| MM08043 | | |
| 2263:K3 | | |
| VP08-35 | ✓ | Micromorphology without remark. |
| MM08043 | | |
| 2263:K4 | | |
| VP08-35 | ✓ | Micromorphology without remark. |
| MM08043 | | |
| 2264:K1 | | |
| VP08-35 | ✓ | Micromorphology without remark. |
| MM08043 | | |
| 2264:K2 | | |
| VP08-35 | ✓ | Micromorphology without remark. |
| MM08043 | | |
| 2264:K3 | | |
| VP08-35 | ✓ | Micromorphology without remark. |
| MM08043 | | |
| 2264:K4 | | |
| VP08-35 | ✓ | Micromorphology without remark. |
| MM08043 | | |
| 2265:K1 | | |
| VP08-35 | ✓ | Micromorphology without remark. |
| MM08043 | | |
| 2265:K2 | | |
| (2 slides) | | |
| VP08-35 | ✓ | Fresh hemorrhage in the dorsal part |
| MM08043 | | nasal cavity. The olfactory epithelium |
| 2265:K3 | | shows oedema with detachment of the |
| | | epithelial cells (most pronounced in the lowest |
| | | part of the cavity) and is covered by a thin |
| | | layer of serous fluid. |
| VP08-35 | ✓ | Micromorphology without remark. |
| MM08043 | | |
| 2265:K4 | | |
| VP08-35 | ✓ | Micromorphology without remark. |
| MM08043 | | |
| 2266:K1 | | |
| VP08-35 | ✓ | Micromorphology without remark. |
| MM08043 | | |
| 2266:K2 | | |
| VP08-35 | ✓ | Micromorphology without remark. |
| MM08043 | | |
| 2266:K3 | | |
| VP08-35 | ✓ | Micromorphology without remark. |
| MM08043 | | |
| 2266:K4 | | |
| VP08-35 | ✓ | Micromorphology without remark. |
| MM08043 | | |
| 2267:K1 | | |
| VP08-35 | ✓ | Micromorphology without remark. |
| MM08043 | | |
| 2267:K2 | | |
| VP08-35 | ✓ | Fresh hemorrhage in the dorsal part |
| MM08043 | | nasal cavity. The olfactory epithelium shows |
| 2267:K3 | | oedema with detachment of the epithelial |
| | | cells (most pronounced in the lowest part of |
| | | the cavity) and is covered by a thin layer of |
| | | serous fluid. |
| VP08-35 | ✓ | Micromorphology without remark. |
| MM08043 | | |
| 2267:K4 | | |
| VP08-35 | ✓ | Micromorphology without remark. |
| MM08043 | | |
| 2305:K1 | | |
| VP08-35 | ✓ | Micromorphology without remark. |
| MM08043 | | |
| 2305:K2 | | |
| VP08-35 | ✓ | Micromorphology without remark. |
| MM08043 | | |
| 2305:K3 | | |
| VP08-35 | ✓ | Micromorphology without remark. |
| MM08043 | | |
| 2305:K4 | | |
| VP08-35 | ✓ | Micromorphology without remark. |
| MM08043 | | |
| 2306:K1 | | |
| VP08-35 | ✓ | Micromorphology without remark. |
| MM08043 | | |
| 2306:K2 | | |
| VP08-35 | ✓ | Micromorphology without remark. |
| MM08043 | | |
| 2306:K3 | | |
| VP08-35 | ✓ | Micromorphology without remark. |
| MM08043 | | |
| 2306:K4 | | |
| VP08-35 | ✓ | Micromorphology without remark. |
| MM08043 | | |
| 2307:K1 | | |
| VP08-35 | ✓ | Micromorphology without remark. |
| MM08043 | | |
| 2307:K2 | | |
| VP08-35 | ✓ | Micromorphology without remark. |
| MM08043 | | |
| 2307:K3 | | |
| VP08-35 | ✓ | Micromorphology without remark. |
| MM08043 | | |
| 2307:K4 | | |
| VP08-35 | ✓ | Micromorphology without remark. |
| MM08043 | | |
| 2308:K1 | | |
| VP08-35 | ✓ | Micromorphology without remark. |
| MM08043 | | |
| 2308:K2 | | |
| VP08-35 | ✓ | Micromorphology without remark. |
| MM08043 | | |
| 2308:K3 | | |
| VP08-35 | ✓ | Micromorphology without remark. |
| MM08043 | | |
| 2308:K4 | | |
| VP08-35 | ✓ | Micromorphology without remark. |
| MM08043 | | |
| 2309:K1 | | |
| VP08-35 | ✓ | Micromorphology without remark. |
| MM08043 | | |
| 2309:K2 | | |
| VP08-35 | ✓ | Micromorphology without remark. |
| MM08043 | | |
| 2309:K3 | | |
| VP08-35 | ✓ | Micromorphology without remark. |
| MM08043 | | |
| 2309:K4 | | |
| VP08-35 | ✓ | Micromorphology without remark. |
| MM08043 | | |
| 2310:K1 | | |
| VP08-35 | ✓ | Micromorphology without remark. |
| MM08043 | | |
| 2310:K2 | | |
| VP08-35 | ✓ | Micromorphology without remark. |
| MM08043 | | |
| 2310:K3 | | |
| VP08-35 | ✓ | Micromorphology without remark. |
| MM08043 | | |
| 2310:K4 | | |

Group 5

| Slide labeling | Check | Analysis |
|---|---|---|
| VP08-35 | ✓ | Micromorphology without remark. |
| MM08043 | | |
| 2268:K1 | | |
| VP08-35 | ✓ | Micromorphology without remark. |
| MM08043 | | |
| 2268:K2 | | |
| VP08-35 | ✓ | Micromorphology without remark. |
| MM08043 | | |
| 2268:K3 | | |
| VP08-35 | ✓ | Micromorphology without remark. |
| MM08043 | | |
| 2268:K4 | | |
| VP08-35 | ✓ | Micromorphology without remark. |
| MM08043 | | |
| 2269:K1 | | |
| (2 slides) | | |
| VP08-35 | ✓ | Micromorphology without remark. |
| MM08043 | | |
| 2269:K2 | | |
| VP08-35 | ✓ | Micromorphology without remark. |
| MM08043 | | |
| 2269:K3 | | |
| VP08-35 | ✓ | Micromorphology without remark. |
| MM08043 | | |
| 2269:K4 | | |
| VP08-35 | ✓ | Micromorphology without remark. |
| MM08043 | | |
| 2270:K1 | | |
| VP08-35 | ✓ | Micromorphology without remark. |
| MM08043 | | |
| 2270:K2 | | |
| VP08-35 | ✓ | Micromorphology without remark. |
| MM08043 | | |
| 2270:K3 | | |
| VP08-35 | ✓ | Micromorphology without remark. |
| MM08043 | | |
| 2270:K4 | | |
| VP08-35 | ✓ | Micromorphology without remark. |
| MM08043 | | |
| 2271:K1 | | |
| VP08-35 | ✓ | Micromorphology without remark. |
| MM08043 | | |
| 2271:K2 | | |
| VP08-35 | ✓ | Micromorphology without remark. |
| MM08043 | | |
| 2271:K3 | | |
| VP08-35 | ✓ | Micromorphology without remark. |
| MM08043 | | |
| 2271:K4 | | |
| VP08-35 | ✓ | Micromorphology without remark. |
| MM08043 | | |
| 2272:K1 | | |
| VP08-35 | ✓ | Micromorphology without remark. |
| MM08043 | | |
| 2272:K2 | | |
| VP08-35 | ✓ | Micromorphology without remark. |
| MM08043 | | |
| 2272:K3 | | |
| VP08-35 | ✓ | Micromorphology without remark. |
| MM08043 | | |
| 2272:K4 | | |
| VP08-35 | ✓ | Micromorphology without remark. |
| MM08043 | | |
| 2273:K1 | | |
| VP08-35 | ✓ | Micromorphology without remark. |
| MM08043 | | |
| 2273:K2 | | |
| VP08-35 | ✓ | Micromorphology without remark. |
| MM08043 | | |
| 2273:K3 | | |
| (2 slides) | | |
| VP08-35 | ✓ | Micromorphology without remark. |
| MM08043 | | |
| 2273:K4 | | |
| VP08-35 | ✓ | Micromorphology without remark. |
| MM08043 | | |
| 2311:K1 | | |
| VP08-35 | ✓ | Micromorphology without remark. |
| MM08043 | | |
| 2311:K2 | | |
| VP08-35 | ✓ | Fresh hemorrhage in the nasal cavity. |
| MM08043 | | |
| 2311:K3 | | |
| VP08-35 | ✓ | Micromorphology without remark. |
| MM08043 | | |
| 2311:K4 | | |
| VP08-35 | ✓ | Micromorphology without remark. |
| MM08043 | | |
| 2312:K1 | | |
| VP08-35 | ✓ | Micromorphology without remark. |
| MM08043 | | |
| 2312:K2 | | |
| VP08-35 | ✓ | Micromorphology without remark. |
| MM08043 | | |
| 2312:K3 | | |
| VP08-35 | ✓ | Micromorphology without remark. |
| MM08043 | | |
| 2312:K4 | | |
| VP08-35 | ✓ | Micromorphology without remark. |
| MM08043 | | |
| 2313:K1 | | |
| VP08-35 | ✓ | Micromorphology without remark. |
| MM08043 | | |
| 2313:K2 | | |
| VP08-35 | ✓ | Micromorphology without remark. |
| MM08043 | | |
| 2313:K3 | | |
| VP08-35 | ✓ | Micromorphology without remark. |
| MM08043 | | |
| 2313:K4 | | |
| VP08-35 | ✓ | Micromorphology without remark. |
| MM08043 | | |
| 2314:K1 | | |
| (2 slides) | | |
| VP08-35 | ✓ | Micromorphology without remark. |
| MM08043 | | |
| 2314:K2 | | |
| VP08-35 | ✓ | Micromorphology without remark. |
| MM08043 | | |
| 2314:K3 | | |
| VP08-35 | ✓ | Micromorphology without remark. |
| MM08043 | | |
| 2314:K4 | | |
| VP08-35 | ✓ | Micromorphology without remark. |
| MM08043 | | |
| 2315:K1 | | |
| VP08-35 | ✓ | Micromorphology without remark. |
| MM08043 | | |
| 2315:K2 | | |
| VP08-35 | ✓ | Micromorphology without remark. |
| MM08043 | | |
| 2315:K3 | | |
| VP08-35 | ✓ | Micromorphology without remark. |
| MM08043 | | |
| 2315:K4 | | |
| VP08-35 | ✓ | Micromorphology without remark. |
| MM08043 | | |
| 2316:K1 | | |
| VP08-35 | ✓ | Micromorphology without remark. |
| MM08043 | | |
| 2316:K2 | | |
| VP08-35 | ✓ | Micromorphology without remark. |
| MM08043 | | |
| 2316:K3 | | |
| VP08-35 | ✓ | Micromorphology without remark. |
| MM08043 | | |
| 2316:K4 | | |

Group 6

| Slide labeling | Check | Analysis |
|---|---|---|
| VP08-35 | ✓ | Micromorphology without remark |
| MM08043 | | |
| 2274:K1 | | |
| VP08-35 | ✓ | Micromorphology without remark |
| MM08043 | | |
| 2274:K2 | | |
| VP08-35 | ✓ | Micromorphology without remark |
| MM08043 | | |
| 2274:K3 | | |
| VP08-35 | ✓ | Micromorphology without remark |
| MM08043 | | |
| 2274:K4 | | |
| VP08-35 | ✓ | Micromorphology without remark |
| MM08043 | | |
| 2275:K1 | | |
| VP08-35 | ✓ | Micromorphology without remark |
| MM08043 | | |
| 2275:K2 | | |
| VP08-35 | ✓ | Micromorphology without remark. Artefactual rift in specimen. |
| MM08043 | | |
| 2275:K3 | | |
| VP08-35 | ✓ | Micromorphology without remark |
| MM08043 | | |
| 2275:K4 | | |
| VP08-35 | ✓ | Micromorphology without remark |
| MM08043 | | |
| 2276:K1 | | |
| VP08-35 | ✓ | Fresh hemorrhage in the nasal cavity. |
| MM08043 | | |
| 2276:K2 | | |
| VP08-35 | ✓ | Fresh hemorrhage in the dorsal part nasal cavity. The olfactory |
| MM08043 | | epithelium shows oedema with detachment of the epithelial cells |
| 2276:K3 | | (most pronounced in the lowest part of the cavity) and is covered |
| | | by a thin layer of serous fluid. |
| VP08-35 | ✓ | Fresh hemorrhage in the nasal cavity. The olfactory epithelium |
| MM08043 | | shows oedema with detachment of the epithelial cells (most |
| 2276:K4 | | pronounced in the lowest part of the cavity) and is covered by a |
| | | thin layer of serous fluid. |
| VP08-35 | ✓ | Micromorphology without remark |
| MM08043 | | |
| 2277:K1 | | |
| VP08-35 | ✓ | Micromorphology without remark |
| MM08043 | | |
| 2277:K2 | | |
| VP08-35 | ✓ | Micromorphology without remark |
| MM08043 | | |
| 2277:K3 | | |
| VP08-35 | ✓ | Micromorphology without remark |
| MM08043 | | |
| 2277:K4 | | |
| VP08-35 | ✓ | Micromorphology without remark |
| MM08043 | | |
| 2278:K1 | | |
| VP08-35 | ✓ | Micromorphology without remark |
| MM08043 | | |
| 2278:K2 | | |
| VP08-35 | ✓ | Micromorphology without remark |
| MM08043 | | |
| 2278:K3 | | |
| VP08-35 | ✓ | Micromorphology without remark |
| MM08043 | | |
| 2278:K4 | | |
| VP08-35 | ✓ | Micromorphology without remark |
| MM08043 | | |
| 2279:K1 | | |
| (2 slides) | | |
| VP08-35 | ✓ | Micromorphology without remark |
| MM08043 | | |
| 2279:K2 | | |
| VP08-35 | ✓ | Fresh hemorrhage in the dorsal part nasal cavity. The olfactory |
| MM08043 | | epithelium shows oedema with detachment of the epithelial cells |
| 2279:K3 | | (most pronounced in the lowest part of the cavity) and is covered |
| | | by a thin layer of serous fluid. |
| VP08-35 | ✓ | Micromorphology without remark |
| MM08043 | | |
| 2279:K4 | | |
| VP08-35 | ✓ | Micromorphology without remark |
| MM08043 | | |
| 2317:K1 | | |
| VP08-35 | ✓ | Micromorphology without remark |
| MM08043 | | |
| 2317:K2 | | |
| VP08-35 | ✓ | Micromorphology without remark |
| MM08043 | | |
| 2317:K3 | | |
| VP08-35 | ✓ | Micromorphology without remark |
| MM08043 | | |
| 2317:K4 | | |
| VP08-35 | ✓ | Micromorphology without remark |
| MM08043 | | |
| 2318:K1 | | |
| (2 slides) | | |
| VP08-35 | ✓ | Micromorphology without remark |
| MM08043 | | |
| 2318:K2 | | |
| VP08-35 | ✓ | Fresh hemorrhage in the dorsal part nasal cavity. The olfactory |
| MM08043 | | epithelium shows oedema with detachment of the epithelial cells |
| 2318:K3 | | (most pronounced in the lowest part of the cavity) and is covered |
| | | by a thin layer of serous fluid. |
| VP08-35 | ✓ | Micromorphology without remark |
| MM08043 | | |
| 2318:K4 | | |
| VP08-35 | ✓ | Micromorphology without remark |
| MM08043 | | |
| 2319:K1 | | |
| VP08-35 | ✓ | Micromorphology without remark |
| MM08043 | | |
| 2319:K2 | | |
| (2 slides) | | |
| VP08-35 | ✓ | Fresh hemorrhage in the dorsal part nasal cavity. The olfactory |
| MM08043 | | epithelium shows oedema with detachment of the epithelial cells |
| 2319:K3 | | (most pronounced in the lowest part of the cavity) and is covered |
| | | by a thin layer of serous fluid. |
| VP08-35 | ✓ | Micromorphology without remark |
| MM08043 | | |
| 2319:K4 | | |
| VP08-35 | ✓ | Micromorphology without remark |
| MM08043 | | |
| 2320:K1 | | |
| VP08-35 | ✓ | Micromorphology without remark |
| MM08043 | | |
| 2320:K2 | | |
| VP08-35 | ✓ | Micromorphology without remark |
| MM08043 | | |
| 2320:K3 | | |
| VP08-35 | ✓ | Micromorphology without remark |
| MM08043 | | |
| 2320:K4 | | |
| VP08-35 | ✓ | Micromorphology without remark |
| MM08043 | | |
| 2321:K1 | | |
| VP08-35 | ✓ | Micromorphology without remark |
| MM08043 | | |
| 2321:K2 | | |
| VP08-35 | ✓ | Fresh hemorrhage in the dorsal part nasal cavity. The olfactory |
| MM08043 | | epithelium shows oedema with detachment of the epithelial cells |
| 2321:K3 | | (most pronounced in the lowest part of the cavity) and is covered |
| | | by a thin layer of serous fluid. |
| VP08-35 | ✓ | Micromorphology without remark |
| MM08043 | | |
| 2321:K4 | | |
| VP08-35 | ✓ | Micromorphology without remark |
| MM08043 | | |
| 2322:K1 | | |
| VP08-35 | ✓ | Micromorphology without remark |
| MM08043 | | |
| 2322:K2 | | |
| VP08-35 | ✓ | Micromorphology without remark |
| MM00843 | | |
| 2322:K3 | | |
| VP08-35 | ✓ | Micromorphology without remark |
| MM08043 | | |
| 2322:K4 | | |

Group 7

| Slide labeling | Check | Analysis |
|---|---|---|
| VP08-35 | ✓ | Micromorphology without remark |
| MM08043 | | |
| 2280:K1 | | |
| VP08-35 | ✓ | Micromorphology without remark |
| MM08043 | | |
| 2280:K2 | | |
| VP08-35 | ✓ | Micromorphology without remark |
| MM08043 | | |
| 2280:K3 | | |
| VP08-35 | ✓ | Micromorphology without remark |
| MM08043 | | |
| 2280:K4 | | |
| VP08-35 | ✓ | Fresh hemorrhage in the nasal cavity. |
| MM08043 | | |
| 2281:K1 | | |
| VP08-35 | ✓ | Fresh hemorrhage in the nasal cavity. |
| MM08043 | | |
| 2281:K2 | | |
| VP08-35 | ✓ | Fresh hemorrhage in the nasal cavity. |
| MM08043 | | |
| 2281:K3 | | |
| VP08-35 | ✓ | Micromorphology without remark |
| MM08043 | | |
| 2281:K4 | | |
| VP08-35 | ✓ | Micromorphology without remark |
| MM08043 | | |
| 2282:K1 | | |
| VP08-35 | ✓ | Micromorphology without remark |
| MM08043 | | |
| 2282:K2 | | |
| VP08-35 | ✓ | Micromorphology without remark |
| MM08043 | | |
| 2282:K3 | | |
| VP08-35 | ✓ | Micromorphology without remark |
| MM08043 | | |
| 2282:K4 | | |
| VP08-35 | ✓ | Micromorphology without remark |
| MM08043 | | |
| 2283:K1 | | |
| VP08-35 | ✓ | Micromorphology without remark |
| MM08043 | | |
| 2283:K2 | | |
| VP08-35 | ✓ | Micromorphology without remark |
| MM08043 | | |
| 2283:K3 | | |
| VP08-35 | ✓ | Micromorphology without remark |
| MM08043 | | |
| 2283:K4 | | |
| VP08-35 | ✓ | Micromorphology without remark |
| MM08043 | | |
| 2284:K1 | | |
| VP08-35 | ✓ | Micromorphology without remark |
| MM08043 | | |
| 2284:K2 | | |
| VP08-35 | ✓ | Micromorphology without remark |
| MM08043 | | |
| 2284:K3 | | |
| VP08-35 | ✓ | Micromorphology without remark |
| MM08043 | | |
| 2284:K4 | | |
| VP08-35 | ✓ | Micromorphology without remark |
| MM08043 | | |
| 2285:K1 | | |
| VP08-35 | ✓ | Micromorphology without remark |
| MM08043 | | |
| 2285:K2 | | |
| VP08-35 | ✓ | Fresh hemorrhage in the dorsal part |
| MM08043 | | nasal cavity. The olfactory epithelium |
| 2285:K3 | | shows oedema with detachment of the |
| | | epithelial cells (most pronounced in the lowest |
| | | part of the cavity) and is covered by a thin |
| | | layer of serous fluid. |
| VP08-35 | ✓ | Micromorphology without remark |
| MM08043 | | |
| 2285:K4 | | |
| VP08-35 | ✓ | Micromorphology without remark |
| MM08043 | | |
| 2323:K1 | | |
| VP08-35 | ✓ | Minor fresh hemorrhage in the nasal cavity. |
| MM08043 | | |
| 2323:K2 | | |
| VP08-35 | ✓ | Minor fresh hemorrhage in the nasal cavity. |
| MM08043 | | |
| 2323:K3 | | |
| VP08-35 | ✓ | Micromorphology without remark |
| MM08043 | | |
| 2323:K4 | | |
| VP08-35 | ✓ | Micromorphology without remark |
| MM08043 | | |
| 2324:K1 | | |
| VP08-35 | ✓ | Micromorphology without remark |
| MM08043 | | |
| 2324:K2 | | |
| VP08-35 | ✓ | Micromorphology without remark |
| MM08043 | | |
| 2324:K3 | | |
| VP08-35 | ✓ | Micromorphology without remark |
| MM08043 | | |
| 2324:K4 | | |
| VP08-35 | ✓ | Micromorphology without remark |
| MM08043 | | |
| 2325:K1 | | |
| VP08-35 | ✓ | Micromorphology without remark |
| MM08043 | | |
| 2325:K2 | | |
| VP08-35 | ✓ | Micromorphology without remark |
| MM08043 | | |
| 2325:K3 | | |
| VP08-35 | ✓ | Micromorphology without remark |
| MM08043 | | |
| 2325:K4 | | |
| VP08-35 | ✓ | Micromorphology without remark |
| MM08043 | | |
| 2326:K1 | | |
| VP08-35 | ✓ | Fresh hemorrhage in the nasal cavity. |
| MM08043 | | |
| 2326:K2 | | |
| VP08-35 | ✓ | Fresh hemorrhage in the nasal cavity. |
| MM08043 | | |
| 2326:K3 | | |
| VP08-35 | ✓ | Micromorphology without remark |
| MM08043 | | |
| 2326:K4 | | |
| VP08-35 | ✓ | Micromorphology without remark |
| MM08043 | | |
| 2327:K1 | | |
| VP08-35 | ✓ | Micromorphology without remark |
| MM08043 | | |
| 2327:K2 | | |
| VP08-35 | ✓ | Fresh hemorrhage in the nasal cavity. |
| MM08043 | | |
| 2327:K3 | | |
| VP08-35 | ✓ | Micromorphology without remark |
| MM08043 | | |
| 2327:K4 | | |
| VP08-35 | ✓ | Micromorphology without remark |
| MM08043 | | |
| 2328:K1 | | |
| VP08-35 | ✓ | Micromorphology without remark |
| MM08043 | | |
| 2328:K2 | | |
| VP08-35 | ✓ | Micromorphology without remark |
| MM08043 | | |
| 2328:K3 | | |
| VP08-35 | ✓ | Micromorphology without remark |
| MM08043 | | |
| 2328:K4 | | |

Consequently, the frequency of micromorphological alterations is very low in all groups as seen in the tables above. As there is an indicative link between such morphological alterations in the nasal cavity or the olfactory bulb and the occurrence of Bell's Palsy, these results clearly shows that by the use of adjuvants according to the invention it can be expected that the occurrence of Bell's Palsy will be in the same frequency as normally observed (i.e. about 30-40 individuals per 100.000)

1. A method of reducing the incidence of immunization associated Bell's palsy to a frequency not higher than the naturally occurring incidence in a plurality of subjects comprising:
selecting a plurality of subjects to receive a composition that reduces the incidence of immunization associated Bell's palsy to a frequency not higher than the naturally occurring incidence; and
administering to said plurality of subjects a composition that comprises:
i) one or more carboxylic acids,
ii) one or more antigens and, optionally, one or more mono-glycerides.

2-31. (canceled)

32. The method according to claim 1, wherein the one or more carboxylic acids are unbranched, cyclic or acyclic, substituted or unsubstituted alkyl, alkenyl or alkynyl carboxylic acids, optionally having double or triple bonds, which may further optionally be of different kind in the same molecule, wherein the carboxylic acids are from 4 to 30 carbon atoms, from 6 to 24 carbon atoms, from 8 to 20 carbon atoms, or from 12 to 20 carbon atoms.

33. The method according to claim 1, wherein the carboxylic acid is selected from the group consisting of lauric acid, myristic acid, palmitic acid, palmitoleic acid, oleic acid, linoleic acid stearic acid, hexanoic acid, caprylic acid, decanoic acid, capric acid, arachidic acid, behenic acid, lignoceric acid, alpha-linolenic acid, stearidonic acid, eicosapentaenoic acid, docosahexaenoic acid, gamma-linolenic acid, dihomo-gamma-linolenic acid, arachidonic acid, erucic acid, and nervonic acid or any combination thereof.

34. The method according to claim 1, wherein the mono-glycerides are selected from the group consisting of lauric acid, myristic acid, palmitic acid, palmitoleic acid, oleic acid, linoleic acid, stearic acid, hexanoic acid, caprylic acid, decanoic acid, capric acid, arachidic acid, behenic acid, lignoceric acid, alpha-linolenic acid, stearidonic acid, eicosapentaenoic acid, docosahexaenoic acid, gamma-linolenic acid, dihomo-gamma-linolenic acid, arachidonic acid, erucic acid, and nervonic acid or any combination thereof.

35. The method according to claim 1, wherein the concentration of mono-glycerides in said composition is in the range of 0.1 g to 50 g per 100 ml of said composition, 1 g to 20 g per 100 ml of said composition, 0.5 g to 40 g per 100 ml of said composition, 0.5 g to 30 g per 100 ml of said composition, 0.5 g to 25 g per 100 ml of said composition, 1 g to 20 g per 100 ml of said composition, 2 g to 15 g per 100 ml of said composition, or 5 g to 10 g per 100 ml of said composition.

36. The method according to claim 1, wherein the concentration of carboxylic acids in said composition is in the range of 0.1 g to 50 g per 100 ml of said composition, 1 g to 20 g per 100 ml of said composition, 0.5 g to 40 g per 100 ml of said composition, 0.5 g to 30 g per 100 ml of said composition, 0.5 g to 25 g per 100 ml of said composition, 1 g to 20 g per 100 ml of said composition, 2 g to 15 g per 100 ml of said composition, or 5 g to 10 g per 100 ml of said composition.

37. The method according to claim 1, wherein one or more mono-glycerides together with one or more carboxylic acids in said composition is at the most 75% w/v, at the most 50% w/v, at the most 25% w/v, at the most 20% w/v, at the most 15% w/v, at the most 10% w/v, at the most 5% w/v, at the most 4% w/v, at the most 3% w/v, at the most 2% w/v, at the most 1% w/v, at the most 0.5% w/v, or at most 0.1% w/v.

38. The method according to claim 1, wherein the mono-glyceride is mono-olein and the carboxylic acid is oleic acid and/or lauric acid.

39. The method according to claim 1, wherein the composition further comprises a medium.

40. The method according to claim 39, wherein the medium is aqueous.

41. The method according to claim 1, wherein the medium has a pH within the range of pH 4 to pH 9, pH 5 to pH 7, pH 5.5 to pH 6.5, pH 7 to pH 9, or pH 7.5 to pH 8.5, or, wherein the medium has a pH of pH 6, pH 5, or pH 8.

42. The method according to claim 39, wherein the medium further comprises a surface-active agent.

43. The method according to claim 42, wherein the surface-active agent is Pluronic F68 or Pluronic-127.

44. The method according to claim 1, wherein the composition further comprises an adjuvant selected from the group consisting of squalene, soy bean oil, aluminum hydroxide, aluminum phosphate, aluminum hydroxyphosphate sulfate, and aluminum potassium sulfate or any combination thereof.

45. The method according to claim 39, wherein the medium further comprises a buffering agent, a stabilizing agent, an osmotically active agent, a preservative, or a pH adjusting agent or any combinations thereof.

46. The method according to claim 1, wherein the incidence of Bell's palsy is reduced to a frequency lower than 40 in 100,000 subjects that receive said composition.

47. The method according to claim 1, wherein the antigen is selected from bacteria, viruses, parasites, allergies, or cancer antigens or combinations thereof.

48. The method according to claim 1, wherein the antigen is selected from one or more viral, bacterial, or parasitic antigens from the following: hepatitis viruses A, B, C, D and E3, HIV, herpes viruses 1,2, 6 and 7, cytomegalovirus, varicella zoster, papilloma virus, Epstein Barr virus, influenza viruses, para-influenza viruses, adenoviruses, bunya viruses, hanta virus, coxsakie viruses, picorna viruses, rotaviruses, respiratory syncytial viruses, pox viruses, rhinoviruses, rubella virus, papovavirus, mumps virus and measles virus, mycobacteria, pneumocci, aerobic gram negative bacilli, mycoplasma, staphylococcus, streptococcus, salmonellae, chlamydiae, helicobacter pylori, plasmodium, leishmania, trypanosome, toxoplasmosis, schistosoma, or filaria.

49. The method according to claim 1, wherein the antigen is selected from one or more of the following: mycobacteria, pneumococcus, aerobic gram negative bacilli, mycoplasma, staphylococcus, streptococcus, salmonellae, or chlamydiae.

50. The method according to claim 1, wherein the antigen is selected from one or more antigens associated with parasitic malaria, leishmaniasis, trypanosomiasis, toxoplasmosis, schistosomiasis, or filariasis.

51. The method according to claim 1, wherein the antigen is selected from one or more antigens associated with breast cancer, stomach cancer, colon cancer, rectal cancer, cancer of the head and neck, renal cancer, malignant melanoma, laryngeal cancer, ovarian cancer, cervical cancer, or prostate cancer.

52. The method according to claim 1, wherein the antigen is selected from one or more antigens associated with allergies to house dust mites, pollen, or environmental allergens or an autoimmune diseases.

53. The method according to claim 1, wherein the composition comprises an amount of adjuvant and antigen sufficient to elicit an enhanced immune response.

54. An immunogenic composition comprising one or more carboxylic acids, one or more antigens, and one or more mono-glycerides.

55. The immunogenic composition of claim 54, wherein the immunogenic composition is formulated for parenteral or mucosal administration.

56. The immunogenic composition of claim 54, wherein the immunogenic composition is formulated for administration to the mucosa of the nose, mouth, vagina, rectum or intestine.

57. The immunogenic composition of claim 54, wherein the immunogenic composition is formulated for administration to the mucosa of the nasal cavity.

58. The method of claim 1, wherein the composition is administered to the mucosa of the nasal cavity of said subject by spray or droplet.

59. The immunogenic composition of claim 54, wherein the immunogenic composition comprises:
i) from 0.1 g to 90 g carboxylic acid,
ii) from 0.1 g to 90 g monoglyceride, and
iii) from 0.001 to 90 g of antigen
per 100 g of immunogenic composition.

60. The immunogenic composition of claim 54, wherein the carboxylic acid is oleic acid and/or lauric acid and the monoglyceride is mono-olein.

61. The immunogenic composition of claim 54, wherein the immunogenic composition further comprises squalene or soy bean oil or a mixture thereof.

62. The method of claim 1, wherein the naturally occurring incidence of immunization associated Bell's palsy is 30-40 individuals per 100,000 individuals.
